(19) United States
(12) Patent Application Publication
(10) Pub. No.: US 2008/0033441 A1
(43) Pub. Date: Feb. 7, 2008

(54) Ligament graft-fixing apparatus

(75) Inventor: Konsei Shino, Osaka (JP)

Correspondence Address: BUCHANAN, INGERSOLL & ROONEY PC, POST OFFICE BOX 1404, ALEXANDRIA, VA, 22313-1404, US

(73) Assignee: MEIRA CORPORATION, Nagoya-shi (JP)

(21) Appl. No.: 11/066,198

(22) Filed: Feb. 25, 2005

(51) Int. Cl. A61B 17/56; A61B 17/58

(52) U.S. Cl. 606/80; 606/100

(57) ABSTRACT

A ligament graft-fixing apparatus has a rod-shaped shaft and a ligament graft-fixing unit that can be removably mounted on the rod-shaped shaft. The ligament graft-fixing unit has a screw part which drills a bone to form a tunnel therein to hold the ligament graft-fixing unit in the bone tunnel; a ligament graft-holding part; and a mounting tunnel in which the rod-shaped shaft is mounted. The mounting tunnel and the rod-shaped shaft have a torque transmission mechanism which transmits a rotational force imparted to the rod-shaped shaft to the ligament graft-fixing unit; and a removal prevention mechanism which holds the ligament graft-fixing unit on the rod-shaped shaft.

### BACKGROUND OF THE INVENTION

The present invention relates to a ligament graft-fixing apparatus for fixing a transplanted ligament graft to predetermined positions in a bone tunnel.

In fixing the transplanted ligament graft to the predetermined position in the bone tunnel to perform an operation of reconstructing anterior/posterior cruciate or collateral ligament grafts of knee by the conventional operation, both ends of the graft are fixed to the surface of a tibia and that of a femur with an button/screw post with sutures or a staple. However, the shorter the distance between the two fixation sites is, the more advantageous the graft remodeling is because of less bungee cord effect during motion. Thus in recent years, an operation of fixing an end of the graft to a predetermined position inside the bone tunnel has been performed.

Each of the ligament graft-fixing apparatuses for fixing the graft to the predetermined position in the bone tunnel disclosed in U.S. Pat. No. 4,870,957 and U.S. Pat. No. 6,616,694 has the screw member and the ligament graft-holding member formed separately from the screw member. These ligament graft-fixing apparatuses are capable of fixing the ligament graft to the predetermined position in the bone tunnel by placing the screw member at a predetermined position in the bone tunnel and then engaging the ligament graft-fixing stud to which the ligament graft is fixed with the screw member at a predetermined position therein.

However, in these ligament graft-fixing apparatuses, the screw member and the ligament graft-holding member are formed separately. Thus an additional step to engage the ligament graft-holding member with the screw member is necessary during operation. More specifically, it is necessary to place the screw member at the predetermined position and then engage the ligament graft-holding member with the screw member. Consequently it is necessary to perform such a complicated additional work during operation.

The ligament graft-fixing apparatus having the screw member and the ligament graft-fixing tunnel is disclosed in U.S. Pat. No. 5,152,790. There is a fear in this ligament graft-fixing apparatus that the shaft for rotating the ligament graft-fixing unit to advance it comes off from the ligament graft-fixing unit while placing the ligament graft-fixing unit at the predetermined position in the bone tunnel. Thus an extreme attention should be paid during the operation. In both the ligament graft-fixing apparatuses disclosed in U.S. Pat. No. 4,870,957 and U.S. Pat. No. 6,616,694, there is a fear that the shaft comes off from the screw member while placing the screw member at the predetermined position in the bone tunnel.

### SUMMARY OF THE INVENTION

The first invention provides a ligament graft-fixing apparatus comprising: a rod-shaped shaft; and a ligament graft-fixing unit adapted to be removably mounted on the rod-shaped shaft to fix a ligament graft to a bone tunnel, the ligament graft-fixing unit comprising: a screw part provided on a side surface of the ligament graft-fixing unit which is adapted to penetrate into a bone tunnel of a bone by drilling the bone through rotation of the ligament graft-fixing unit to hold the ligament graft-fixing unit in the bone tunnel after the ligament graft-fixing unit penetrates into the bone tunnel; a ligament graft-holding part provided at a rear end portion of the ligament graft-fixing unit to hold one of the ligament graft or a connection member mounted on the ligament graft; and a mounting tunnel for mounting the ligament graft-fixing unit to the rod shaped shaft, the mounting tunnel being formed in the ligament graft-fixing unit from a front end thereof toward a rear end thereof, and wherein the mounting tunnel and the rod-shaped shaft comprise: a torque transmission mechanism which transmits a rotational force imparted to the rod-shaped shaft to the ligament graft-fixing unit when the rod-shaped shaft is rotated in a direction in which the ligament graft-fixing unit advances in the bone tunnel while the ligament graft-fixing unit is mounted on the rod-shaped shaft; and a removal prevention mechanism which holds the ligament graft-fixing unit on the rod-shaped shaft.

The second invention provides a method of fixing a ligament graft in a bone comprising: positioning a one-piece ligament graft-fixing unit at a bone tunnel provided in a bone, the one-piece ligament graft-fixing unit comprising a screw part provided at a side surface and a ligament-graft-holding part holding one of a ligament graft and a connection member mounted on the ligament graft; engaging a shaft with the one-piece ligament graft-fixing unit in a torque transmitting manner and rotating the shaft to rotate the one-piece ligament graft-fixing unit so that the one-piece ligament graft-fixing unit moves along the bone tunnel as the screw part of the one-piece ligament graft-fixing unit drills into the bone to hold the one-piece ligament graft-fixing unit in place within the bone tunnel; and disengaging the shaft from the one-piece ligament graft-fixing unit.

The third invention provides a ligament graft-fixing apparatus comprising: an elongated shaft; a unitary one-piece ligament graft-fixing unit positionable in a bone tunnel of a bone and adapted to be connected to the shaft in a connected state to move the ligament graft-fixing unit along the bone tunnel and disconnected from the shaft in a disconnected state; the ligament graft-fixing unit comprising a mounting tunnel adapted to receive an end of the shaft in the connected state; the shaft and the ligament graft-fixing unit comprising portions which engage one another to hold the ligament graft-fixing unit on the shaft in the connected state and to transmit torque from the shaft to the ligament graft-fixing unit in the connected state upon rotation of the shaft; the ligament graft-fixing unit comprising a ligament graft holding part to hole one of a ligament graft and a connection member mounted on the ligament graft; and the ligament graft-fixing unit comprising a screw part on its outer periphery that drills into the bone of the bone tunnel during rotation of the shaft in the connected state to move the ligament graft-fixing unit along the bone tunnel and hold the ligament graft-fixing unit in the bone tunnel.

### BRIEF DESCRIPTION OF THE DRAWINGS

FIG. 1 is an outlook view showing an embodiment of the ligament graft-fixing apparatus of the present invention.

FIG. 2 is a front view showing a ligament graft-fixing unit constructing the ligament graft-fixing apparatus shown in FIG. 1.

FIG. 3 is a sectional view showing the ligament graft-fixing unit shown in FIG. 2.

FIG. 4 is a plan view showing the ligament graft-fixing unit shown in FIG. 2.

FIG. 5 is a bottom view showing the ligament graft-fixing unit shown in FIG. 2.

FIG. 6 is an enlarged view showing a front portion of a rod-shaped shaft constructing the ligament graft-fixing apparatus shown in FIG. 1.

FIG. 7 is an explanatory view for explaining an engaging mechanism for engaging the ligament graft-fixing unit and the rod-shaped shaft each other.

FIG. 8 is an outlook view showing another embodiment of the ligament graft-fixing apparatus of the present invention.

FIG. 9 is a sectional view showing a ligament graft-fixing unit constructing the ligament graft-fixing apparatus shown in FIG. 8.

FIG. 10 is a plan view showing the ligament graft-fixing unit shown in FIG. 9.

FIG. 11 is a bottom view showing the ligament graft-fixing unit shown in FIG. 9.

FIG. 12 is an enlarged view showing a rod-shaped shaft constructing the ligament graft-fixing apparatus shown in FIG. 8.

FIG. 13 is a sectional view showing the rod-shaped shaft shown in FIG. 12.

FIG. 14 is a plan view showing the rod-shaped shaft shown in FIG. 12.

FIG. 15 is an explanatory view for explaining an engaging mechanism for engaging the ligament graft-fixing unit and the rod-shaped shaft each other.

FIG. 16 is an explanatory view for explaining another embodiment of the ligament graft-fixing apparatus of the present invention.

FIG. 17 is an outlook view showing still another embodiment of the ligament graft-fixing apparatus of the present invention.

FIG. 18 is a front view showing a ligament graft-fixing unit constructing the ligament graft-fixing apparatus shown in FIG. 17.

FIG. 19 is a sectional view showing the ligament graft-fixing unit shown in FIG. 18.

FIG. 20 is a bottom view showing the ligament graft-fixing unit shown in FIG. 18.

FIG. 21 is a plan view showing the ligament graft-fixing unit shown in FIG. 18.

FIG. 22 is an enlarged view showing a front portion of a rod-shaped shaft constructing the ligament graft-fixing apparatus shown in FIG. 17.

FIG. 23 is an explanatory view for explaining an engaging mechanism for engaging the ligament graft-fixing unit and the rod-shaped shaft each other.

FIG. 24 is an outlook view showing still another embodiment of the ligament graft-fixing apparatus of the present invention.

FIG. 25 is a front view showing a ligament graft-fixing unit constructing the ligament graft-fixing apparatus shown in FIG. 24.

FIG. 26 is a sectional view showing the ligament graft-fixing unit shown in FIG. 25.

FIG. 27 is a bottom view showing the ligament graft-fixing unit shown in FIG. 25.

FIG. 28 is a plan view showing the ligament graft-fixing unit shown in FIG. 25.

FIG. 29 is an explanatory view for explaining an engaging mechanism for engaging the ligament graft-fixing unit and the rod-shaped shaft each other.

FIG. 30 shows the rod-shaped shaft and a drill grip mounted thereon.

FIG. 31 is an explanatory view for explaining a method of the present invention for placing the ligament graft-fixing unit at a predetermined position in a bone tunnel.

FIG. 32 is an explanatory view for explaining the method of placing the ligament graft-fixing unit at the predetermined position in the bone tunnel.

FIG. 33 is an explanatory view for explaining the method of placing the ligament graft-fixing unit at the predetermined position in the bone tunnel.

FIG. 34 is an explanatory view for explaining the method of placing the ligament graft-fixing unit at the predetermined position in the bone tunnel.

FIG. 35 is an explanatory view for explaining the method of placing the ligament graft-fixing unit at the predetermined position in the bone tunnel.

FIG. 36 is an outlook view showing an embodiment of a rotary instrument.

FIG. 37 is an enlarged side view showing a front end portion of the rotary instrument shown in FIG. 36.

FIG. 38 is an explanatory view for explaining the method of removing the ligament graft-fixing unit from a bone tunnel to be carried out by using the rotary instrument.

FIG. 39 is an outlook view showing another rotary instrument

FIG. 40 is an explanatory view for explaining the method of removing the. ligament graft-fixing unit from a bone tunnel to be carried out by using the rotary instrument.

### DETAILED DESCRIPTION OF THE PREFERRED EMBODIMENTS

The embodiments of ligament graft-fixing apparatus of the present invention will be described below with reference to the accompanied drawings.

In one embodiment of ligament graft-fixing apparatus of the present invention, there is provided a ligament graft-fixing apparatus including a rod-shaped shaft 3; and a ligament graft-fixing unit 2 adapted to be removably mounted on the rod-shaped shaft 3 to fix a ligament graft 80 to a bone tunnel. The ligament graft-fixing unit 2 includes a screw part 13 provided on a side surface of the ligament graft-fixing unit 2. The screw part 13 is adapted to penetrate into a bone tunnel of a bone by drilling the bone through rotation of the ligament graft-fixing unit to hold the ligament graft-fixing unit 2 in the bone tunnel after the ligament graft-fixing unit 2 penetrates into the bone tunnel. The ligament graft-fixing unit 2 further includes a ligament graft-holding part 8, provided at a rear end portion of the ligament graft-fixing unit 2 to hold one of the ligament graft 80 or a connection member mounted on the ligament graft 80; and a mounting tunnel 9 for mounting the ligament graft-fixing unit 2 to the rod shaped shaft 3, the mounting tunnel 9 being formed in the ligament graft-fixing unit 2 from a front end thereof toward a rear end thereof. The mounting tunnel 9 and the rod-shaped shaft 3 comprise a torque transmission mechanism which transmits a rotational force imparted to the rod-shaped shaft 3 to the ligament graft-fixing unit 2 when the rod-shaped shaft 3 is rotated in a direction in which the ligament graft-fixing unit 2 advances in the bone tunnel while the ligament graft-fixing unit 2 is mounted on the rod-shaped shaft 3; and a removal prevention mechanism which holds said ligament graft-fixing unit 2 on the rod-shaped shaft 3.

The ligament graft-fixing apparatus 1 comprises an elongated shaft 3 and a unitary one-piece ligament graft-fixing unit 2 positionable in a bone tunnel of a bone and adapted to be connected to the shaft 3 in a connected state to move the ligament graft-fixing unit 2 along the bone tunnel and disconnected from the shaft 3 in a disconnected state. The ligament graft-fixing unit 2 comprises a mounting tunnel 9 adapted to receive an end of the shaft 3 in the connected state. The shaft 3 and the ligament graft-fixing unit 2 comprise portions which engage one another to hold the ligament graft-fixing unit 2 on the shaft 3 in the connected state and to transmit torque from the shaft 3 to the ligament graft-fixing unit 2 in the connected state upon rotation of the shaft 3. The ligament graft-fixing unit 2 comprises a ligament graft holding part 8 to hold one of a ligament graft 80 and a connection member mounted on the ligament graft 80. The ligament graft-fixing unit 2 comprises a screw part 13 on its outer periphery that drills into the bone of the bone tunnel during rotation of the shaft 3 in the connected state to move the ligament graft-fixing unit 2 along the bone tunnel and hold the ligament graft-fixing unit 2 in the bone tunnel.

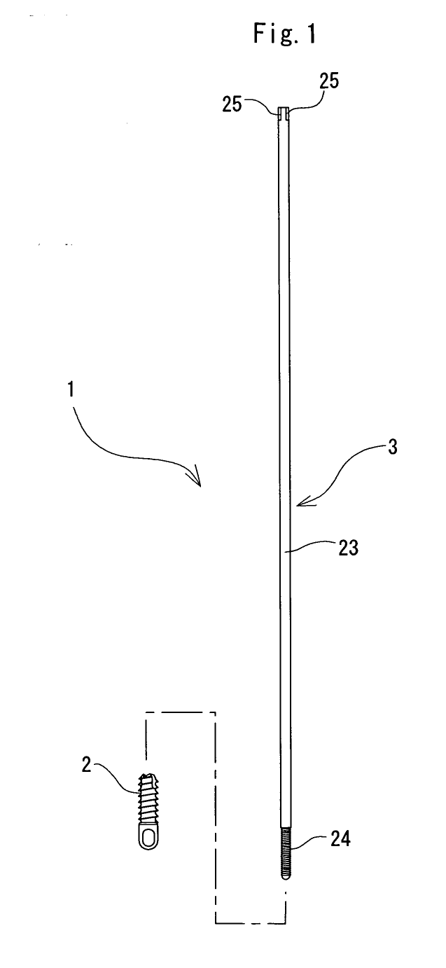

As shown in FIG. 1, the ligament graft-fixing apparatus 1 includes the ligament graft-fixing unit 2 and the rod-shaped shaft (hereinafter referred to as merely shaft) 3.

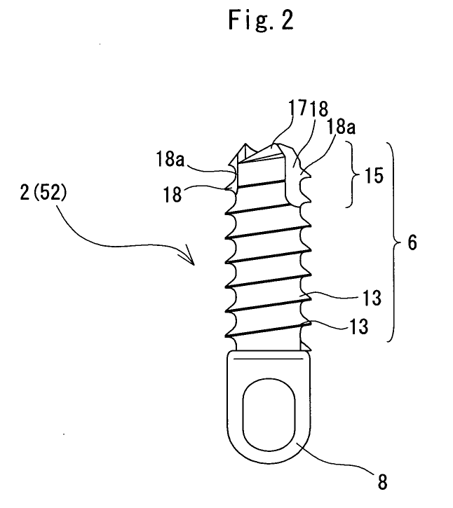

As shown in FIG. 2, the ligament graft-fixing unit 2 has a cylindrical body part 6 whose front end (upper end in the drawings) is open and whose rear end (lower end in the drawings) is closed; the screw part 13 formed on the side surface of the cylindrical body part 6 from a neighborhood of the front end thereof toward the rear end thereof; a cutting-edge part 15 formed at the front portion of the body part 6 to drill a bone and enlarge the diameter of the bone tunnel; the ligament graft-holding part 8 formed at the rear side of the body part 6; and the mounting tunnel 9 formed inside the body part 6.

As shown in FIG. 2, the screw part 13 is formed as a screw thread disposed on the side surface of the body part 6 from a neighborhood of the front end thereof to a neighborhood of the rear end thereof. The height of the screw thread 13 is formed almost uniform except the neighborhood of the front end of the body part 6. The height of the screw thread 13 in the neighborhood of the front end of the body part 6 is set a little smaller than that of the screw thread in other portions of the body part 6. The screw thread 13 is spaced at almost regular intervals. More specifically, the screw thread 13 is spaced at intervals of 1.4 mm to 2.5 mm and preferably 1.5 mm to 2.2 mm. The height of the screw thread 13 is set to 0.4 mm to 1.2 mm and preferably to 0.5 mm to 1.0 mm. The outer diameter of the body part 6 is set to 4.5 mm to 10.0 mm and preferably to 5.0 mm to 7.0 mm. It is preferable to set the diameter of the valley to 3.5 mm to 9.0 mm.

A few kinds of ligament graft-fixing units of the above-described size are preferably prepared so that units corresponding to thickness of harvested grafts can be selected.

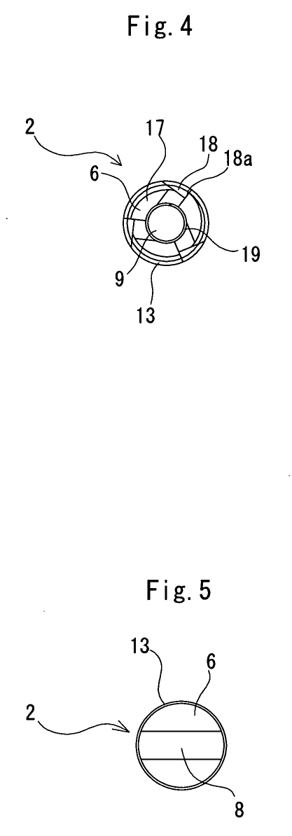

As shown in FIGS. 2 and 4, the cutting-edge part 15 includes a pointed-end portion 17 formed at the front end portion of the body part 6, with the pointed-end portion 17 tapering off toward the front end of the body part 6; and a cut-out portion 18 cut out from the front end of the body part 6 toward its rear end. It is preferable that the cut-out portions 18 are formed almost equiangularly about the axis of the body part 6. In this embodiment, the cut-out portions 18 are formed equiangularly at 120° about the axis of the body part 6. The inclination of the front end of the pointed-end portion 17 (the inclination of the front end of the cylindrical body part 6) is formed almost parallelly with that of the screw thread 13.

In this construction, when the ligament graft-fixing unit 2 is rotated inside the bone tunnel, the inner wall of the bone tunnel is cut by a side edge 18a of the cut-out portion 18. At this time, the ligament graft-fixing unit 2 advances inside the bone tunnel owing to the operation of its screw part 13 with the ligament graft-fixing unit 2 rotating and is then firmly held inside the bone tunnel.

As shown in FIGS. 2 and 5, the ligament graft-holding part 8 is formed at the rear end side of the body part 6 and has a hole for mounting the ligament graft 80 on the ligament graft-holding part 8 directly or indirectly. Concretely the ligament graft-holding part 8 is formed in plate shape and has the hole. Especially, the hole is preferably formed in an ellipse or a circle. The ligament graft 80 is mounted on the ligament graft-holding part 8 directly or indirectly through a connection member (ligature). The ligament graft-holding part may be U-shaped and extends rearward from the rear end portion (lower end in the drawings) of the body part 6. It is preferable that the width of the ligament graft-holding part (U-shaped portion) 8 is formed smaller than the outer diameter of the body part 6 to prevent the ligament graft-holding part 8 from interfering with the advance of the ligament graft-fixing unit 2 in the bone tunnel. The ligament graft-holding part may have any configurations, provided that the ligament graft 80 can be mounted on the ligament graft-holding part.

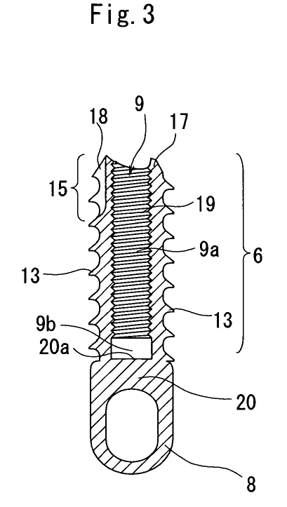

As shown in FIG. 3, the mounting tunnel 9 is composed of an inner tunnel formed inside the body part 6 and a closing part 20.

The mounting tunnel 9 is composed of an accommodation tunnel 9a which is formed from the front end of the body part 6 to the neighborhood of the rear end portion of the body part 6 and which has a mounting tunnel-side engaging part 19, engaging a shaft-side engaging part 27, formed on the inner surface of the accommodation tunnel 9a; and a cylindrical accommodation space 9b, formed rearward from the accommodation tunnel 9a, for accommodating a contact part 28 provided at the front end portion of the shaft 3. A front-end surface (contact surface) 20a of the closing part 20 is formed flatly and contacts only a portion of the contact part 28 of the shaft 3.

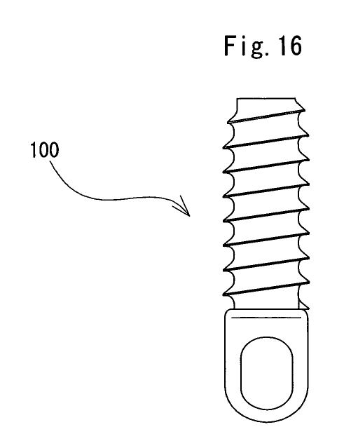

The ligament graft-fixing unit 2 constituting the ligament graft-fixing apparatus has the cutting-edge part 15 on the front portion of the cylindrical body part 6; and the screw part 13 formed on the side surface of the cylindrical body part 6 from a neighborhood of the front end of the body part 6 toward the rear end thereof. Instead of this type, as shown in FIG. 16, it is possible to use a ligament graft-fixing unit 100 not having the cutting-edge part but having the screw part formed on the side surface of the body part from its rear end to its front end whose diameter is a little smaller than that of other portions thereof.

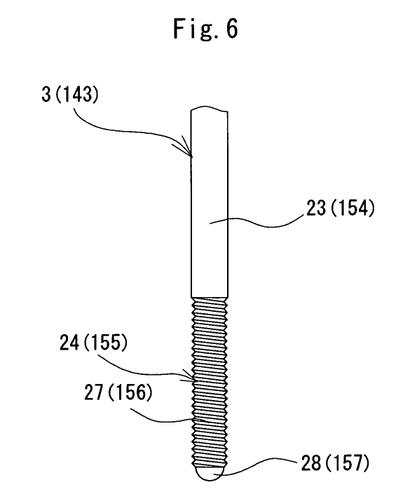

The shaft 3 is solid. As shown in FIGS. 1 and 6, the shaft 3 has a rod-shaped body part 23; an insertion part 24 formed at one side of the body part 23; and a drill-mounting part 25, disposed at the other end portion of the body part 23, on which a rotational force-imparting instrument such as a drill can be mounted.

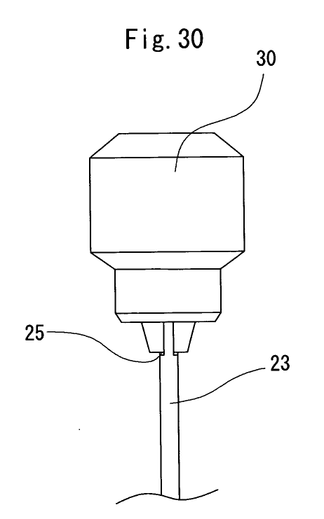

The body part 23 has an almost uniform outer diameter from its one end to its other end. The drill-mounting part 25 is formed by forming a flat surface on the rear end portion of the body part 23 every predetermined (equal) angle about the axis of the body part 23. In this embodiment, the drill-mounting part 25 is formed every 120°. As shown in FIG. 30, the shaft 3 is mounted on a grip 30 of a drill in the drill-mounting part 25.

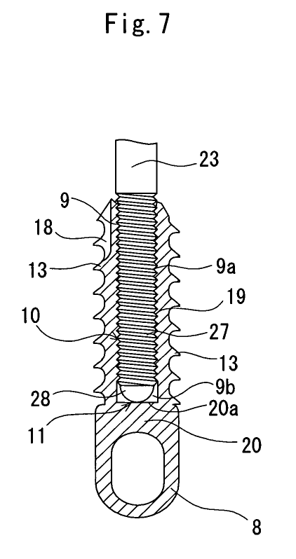

The insertion part 24 has the shaft-side engaging part 27, disposed at one side of the body part 23, which engages the mounting tunnel-side engaging part 19; and the approximately semispherical contact part 28 disposed at a position forward (downward) from the shaft-side engaging part 27, namely, at the front portion of the insertion part 24. The outer diameter of the shaft-side engaging part 27 is set a little smaller than that of the body part 23. The length of the shaft-side engaging part 27 is set a little larger than that of the mounting tunnel-side engaging part 19. As shown in FIG. 7, the shaft 3 is accommodated in the mounting tunnel 9 up to a position thereof in the neighborhood of the rear end portion of the shaft-side engaging part 27 thereof.

The mounting tunnel 9 and the rod-shaped shaft 3 comprise a torque transmission mechanism which transmits a rotational force imparted to the rod-shaped shaft 3 to the ligament graft-fixing unit 2 when the rod-shaped shaft 3 is rotated in a direction in which the ligament graft-fixing unit 2 advances in the bone tunnel while the ligament graft-fixing unit 2 is mounted on the rod-shaped shaft 3; and a removal prevention mechanism which holds the ligament graft-fixing unit 2 on the rod-shaped shaft 3.

The torque transmission mechanism include an engaging mechanism 10, formed on the shaft 3 and the mounting tunnel 9, whose engaging parts engage each other in a direction in which the shaft 3 is rotated to cause the ligament graft-fixing unit 2 to drill the bone; and the torque transmission mechanism also includes a contact mechanism 11 whose parts contact each other in a way preventing the shaft 3 and the mounting tunnel 9 from being tightly fixed to each other. The removal prevention mechanism comprises the engaging mechanism 10.

As shown in FIG. 7, the engaging mechanism 10 has the shaft-side engaging part 27 disposed at the front end portion of the shaft 3; and the mounting tunnel-side engaging part 19 formed on the inner surface of the mounting tunnel 9. The shaft-side engaging part 27 and the mounting tunnel-side engaging part 19 are threaded in a direction in which the shaft-side engaging part 27 and the mounting tunnel-side engaging part 19 engage each other, when the shaft 3 is rotated in the mounting tunnel 9 in the bone-drilling direction.

As shown in FIG. 7, the contact mechanism 11 has the contact surface 20a formed in the mounting tunnel 9; and the contact part 28 disposed at the front end portion of the shaft 3 which contacts only a portion of the contact surface 20a. The contact surface 20a and the contact part 28 contact each other in a way preventing the rod-shaped shaft 3 and the mounting tunnel 9 from being tightly fixed to each other.

The contact part 28 is formed as a rounded part 28 disposed forward from the shaft-side engaging part 27 disposed at the front end portion of the shaft 3. The contact surface 20a is disposed rearward from the mounting tunnel-side engaging part 19 formed on the inner surface of the mounting tunnel 9, thus closing the rear end of the mounting tunnel 9. It is preferable that the rounded part 28 is formed approximately semispherically or approximately semi-elliptically. In this embodiment, the rounded part 28 is formed approximately semispherically. Owing to contact between the front-end surface of the contact part 28 and the closing surface 20a, the shaft 3 is loosely fixed to the mounting tunnel 9. Thus the shaft 3 can be easily removed from the ligament graft-fixing unit 2.

In above-described construction, the shaft 3 inserted into the mounting tunnel 9 advances, with the shaft-side engaging part 27 in engagement with the mounting tunnel-side engaging part 19 until the front end of the rounded part 28 disposed at the front end portion of the shaft 3 and the closing surface 20a contact each other. Owing to the contact therebetween, the shaft 3 is fixed to the mounting tunnel 9. Thereafter the shaft 3 is rotated in its advance direction. As a result, a rotational force imparted to the shaft 3 is transmitted to the ligament graft-fixing unit 2. Consequently the ligament graft-fixing unit 2 advances in the bone tunnel toward its front end (cutting-edge part 15), while the ligament graft-fixing unit 2 is rotating in the bone-drilling direction. After the ligament graft-fixing unit 2 is placed at a predetermined position in the bone tunnel, the shaft 3 is rotated in a reverse direction. As a result, the shaft-side engaging part 27 and the mounting tunnel-side engaging part 19 disengage from each other. Thereby the shaft 3 can be removed from the mounting tunnel 9. As described above, the engaging mechanism and the contact mechanism constitute the torque transmission mechanism, and the engaging mechanism constitutes the removal prevention mechanism.

The torque transmission mechanism and the removal prevention mechanism may be composed of a locking mechanism and the contact mechanism instead of the engaging mechanism and the contact mechanism. Similarly to the above-described engaging mechanism 10, it is preferable that the locking mechanism has a shaft-side locking part and a mounting tunnel-side locking part and that when the shaft is rotated in the mounting tunnel in the bone-drilling direction, the mounting tunnel-side locking part of the mounting tunnel and the shaft-side locking part of the shaft engage each other. Preferably the contact mechanism is so constructed that the front end portion of the shaft contacts only a portion of the inner surface of the mounting tunnel to prevent the shaft from being tightly fixed to the ligament graft-fixing unit.

A grip of a rotational force-imparting instrument such as a drill may be directly mounted on the body part 23 instead of the drill-mounting part 25. The other end portion of the body part 23 may have a shape of drill cutting edge without having the drill-mounting part at the other end portion thereof.

Any materials can be used for the ligament graft-fixing unit 2 and the shaft 3, provided that they have a certain degree of strength and a favorable suitability to a living body. For example, it is possible to use stainless steel (SUS304, SUS316 of JISG4304), pure titanium (JISH4670), and titanium alloy (Ti-6A1-4V of JISH4657, ASTM F-136 Ti-6A1-4V ELI).

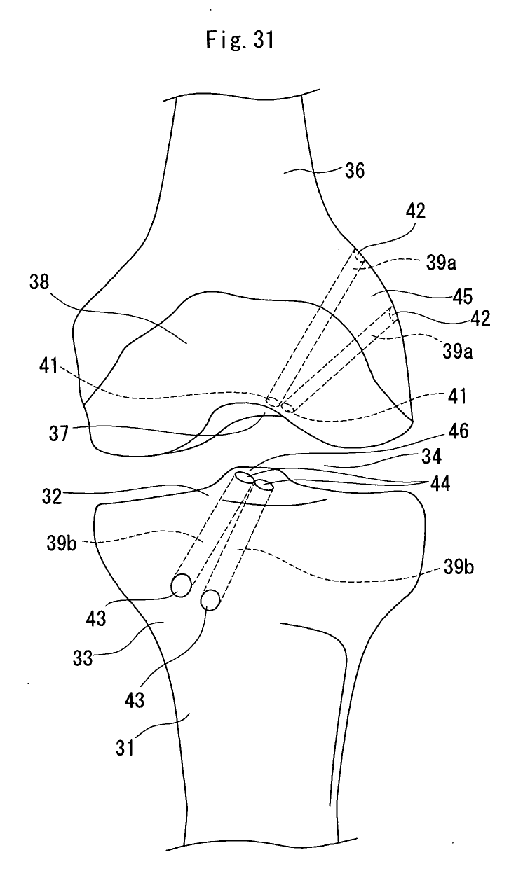

A method of placing the ligament graft-fixing apparatus of the above-described embodiment at a predetermined position in a bone tunnel(a method of fixing the ligament graft in a bone) is described below by exemplifying an operation for reconstructing an anterior cruciate ligament graft of knee with a bundle of double ligament FIGS. 31, 32, 33, and 34 are an explanatory view for explaining the method of placing the ligament graft-fixing unit at a predetermined position in the bone tunnel. FIGS. 31, 32, 33 and 34 show a front view of a left leg.

The method of fixing the ligament graft in a bone comprises: positioning a one-piece ligament graft-fixing unit 2 at a bone tunnel 39a provided in a bone, the one-piece ligament graft-fixing unit 2 comprising a screw part 13 provided at a side surface and a ligament-graft-holding part 8 holding one of a ligament graft 80 and a connection member 82 mounted on the ligament graft 80; engaging a shaft 3 with the one-piece ligament graft-fixing unit 2 in a torque transmitting manner and rotating the shaft 3 to rotate the one-piece ligament graft-fixing unit 2 so that the one-piece ligament graft-fixing unit 2 moves along the bone tunnel 39a as the screw part 13 of the one-piece ligament graft-fixing unit 2 drills into the bone to hold the one-piece ligament graft-fixing unit 2 in place within the bone tunnel 39a; and disengaging the shaft 3 from the one-piece ligament graft-fixing unit 2.

It is preferable that the ligament graft-fixing unit 2 includes a mounting tunnel 9 provided in the one-piece ligament graft-fixing unit 2, the shaft 3 engages the one-piece ligament graft-fixing unit 2 by mounting an end portion of the shaft 3 in the mounting tunnel 9.

It is preferable that the one-piece ligament graft-fixing unit 2 is positioned at one end of the bone tunnel 39a and the shaft 3 is inserted into the bone tunnel 39a from an opposite end of the bone tunnel 39a to engage the one-piece ligament graft-fixing unit 2.

It is preferable that the ligament-graft-holding part 8 is positioned closer to one end of the ligament graft-fixing unit 2 than on opposite end of the ligament graft-fixing unit 2, and the ligament graft-fixing unit 2 is rotated by inserting a portion of the shaft 3 into a mounting tunnel 9 of the ligament graft-fixing unit 2 from the opposite end of the ligament graft-fixing unit 2.

Concretely, as shown in FIG. 31, a side surface 45 of a femur 36 is cut to pierce two guiding steel wires (not shown) into the cut portion and the wires are inserted from the cut portion toward a portion 37 of a femur 36 where an anterior cruciate ligament grafts of knee should be secured. Thereafter by using a drill (3.0 mm-5.0 mm) for forming bone tunnels, having the outer diameter corresponding to a thickness of a harvested ligament graft, two bone tunnels 39a of the femur side having an inner diameter of about 4.5 mm are formed in the femur 36 with the drill guided by the guiding steel wires.

Then a position downward (anterior surface 33 of tibia 31) from an articular surface 32 of the tibia 31 is cut to pierce two guiding steel wires (not shown) into the cut portion and the wires are inserted from the cut portion toward a portion 46 of the tibia 31 where an anterior cruciate ligament graft of knee should be secured.

Thereafter by using a drill (3.0 mm-5.0 mm) for forming tunnels, having an outer diameter corresponding to a thickness of a ligament graft, two bone tunnels 39b of tibia 31 having an inner diameter of about 4.5 mm are formed in the tibia 31 with the drill guided by the guiding steel wires.

In the method of placing the ligament graft-fixing unit, the unit 2 corresponding to a thickness of a ligament graft is selected in advance. Connection members (for example, ligature) 82 are mounted on one end of a transplanted ligament graft 80 and other end thereof respectively. The ligament graft is threaded through a ligament graft-holding part 8 of the unit 2, and the ligament graft is folded in half. Thus a bundle of double ligament is formed.

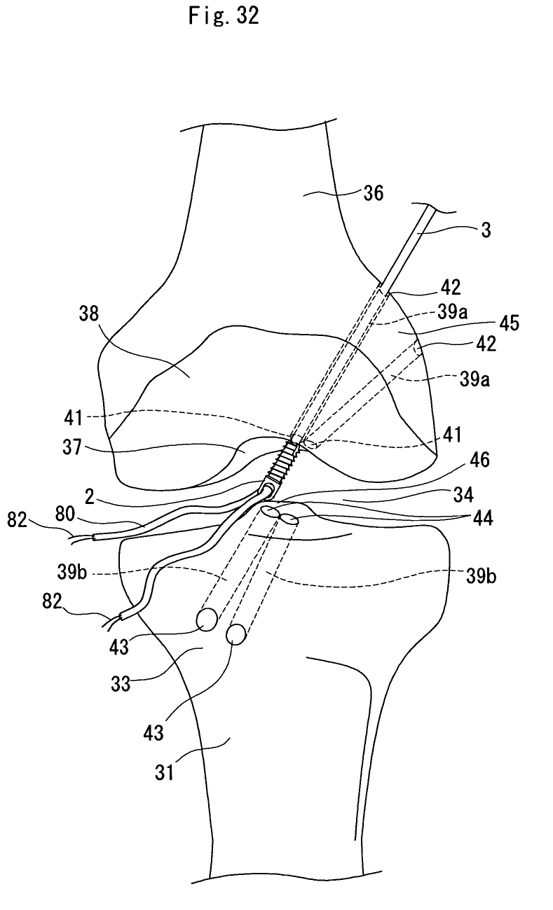

Thereafter the shaft 3 is inserted into the bone tunnels 39a from an opening 42 at the side surface 45 of the femur 36, with the insertion part 24 of the shaft 3 turned downward. Thereafter the front side of the shaft 3 is projected from an opening 41 formed at the femur-side position where the anterior cruciate ligament graft should be secured. After the ligament graft-fixing unit 2 is mounted on the front end portion of the shaft 3, the shaft 3 is moved upward (toward femur) to a position where the ligament graft-fixing unit 2 reach the opening 41, as shown in FIG. 32.

After the rear end portion (drill-mounting part 25) of the shaft 3 is mounted on a drill, the shaft 3 is rotated in a direction in which the ligament graft-fixing unit 2 moves upward, namely, in a direction in which the cutting-edge part is capable of drilling the bone. As a result, the ligament graft-fixing unit 2 advances upward in the bone tunnel 39a, with the ligament graft-fixing unit 2 drilling the inner wall thereof. The drill is rotated until the ligament graft-fixing unit 2 reaches the predetermined portion 40 where the ligament graft-fixing unit 2 should be placed. The shaft 3 may be rotated manually.

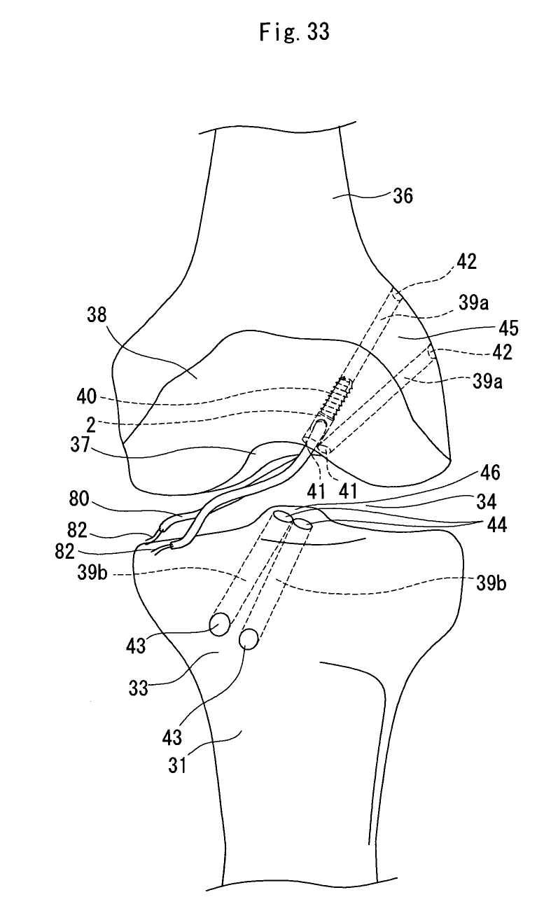

After the ligament graft-fixing unit 2 is placed at the portion 40 where the ligament graft-fixing unit 2 should be placed, the shaft 3 is rotated in the opposite direction to disengage the mounting tunnel-side engaging part 19 and shaft-side engaging part 27 from each other. Thereafter the shaft 3 is pulled out of the bone tunnel 39a. At this time, as shown in FIG. 33, the whole other end of the ligament graft is in the knee joint or a part of the other end of the ligament graft is projected outside the body in a certain length from the cut portion.

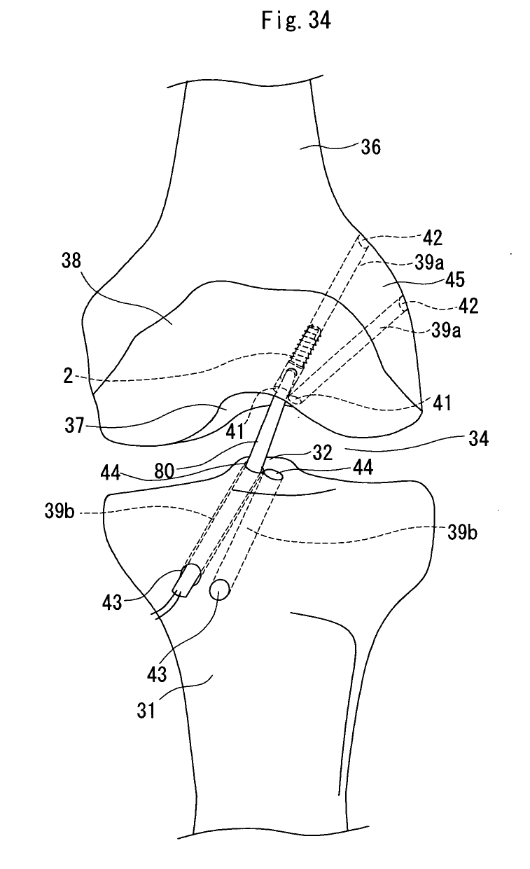

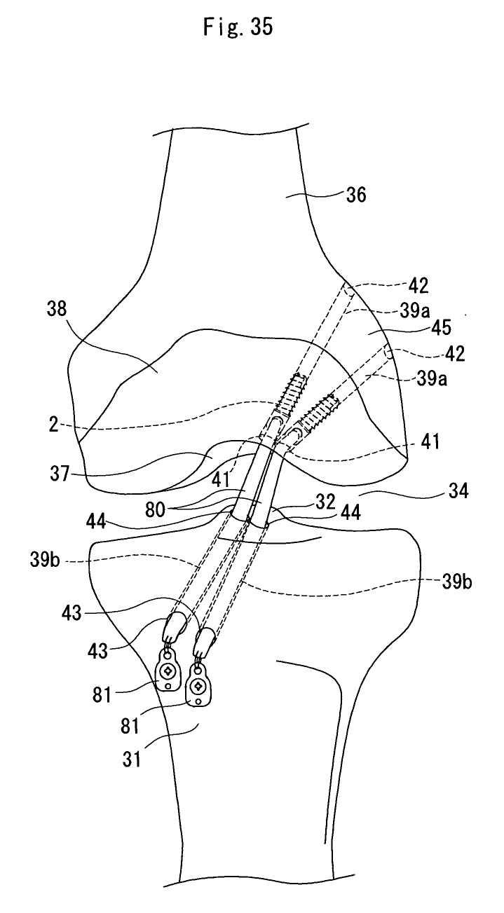

As shown in FIG. 34, after the twisted state of the ligament graft 80 is returned to its original state, the other end of the ligament graft is projected from the opening 43 at the anterior surface-side of the tibia 31 by advancing the ligament graft 80 from the opening 44 through the bone tunnel 39b. Another ligament graft is fixed to the bone tunnel 39a of the femur 36 at the one end of the ligament graft and the other end of the ligament graft is projected from the opening 43 as above-described. As shown in FIG. 35, any tensile force is applied to the connection members 82, and the ligament grafts are fixed to the tibia 31 with the tensile force kept applying thereto. Then the other end of the ligament grafts is fixed to the tibia 31 with an implant (a fixing instrument). Any implants (fixing instruments) being sold can be used for fixing the other end of the ligament graft to the tibia 31.

By using above-described method, the one end of the ligament graft is fixed to the inside femur 36 and the other end of the ligament graft is fixed to the anterior surface 33 of the tibia 31

The ligament graft-fixing unit 2 can be used in the operation for reconstructing anterior crucial ligament graft of knee for single ligament graft (not folded ligament) in the same way as reconstructing the anterior crucial ligament graft of knee with two bundles of doubled tendon graft.

Above-described method of forming bone tunnels 39a inside the femur 36 and bone tunnels 39b inside the tibia 31 is an example. The bone tunnels may be formed by advancing the unit 2 from the femur-side position in the joint of the knee where the anterior cruciate ligament graft should be secured toward the side surface of the femur 36.

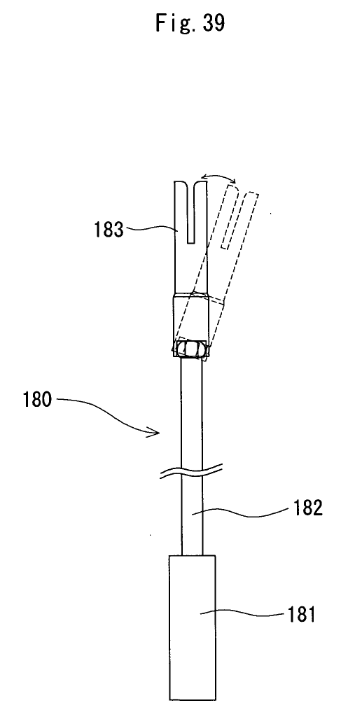

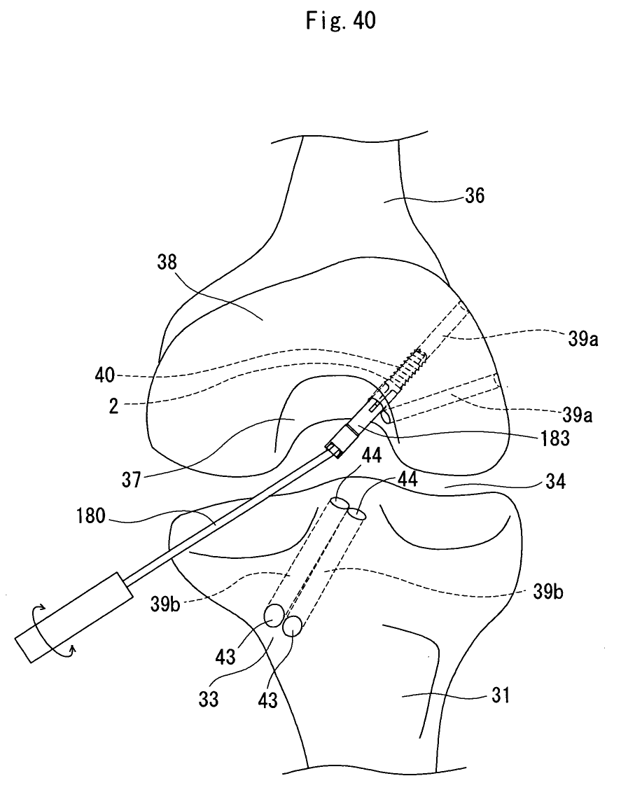

In an reoperation (recovery) for reconstructing anterior or posterior crucial ligament graft of knee a method of pulling the ligament graft-fixing unit 2 out will be described below. FIG. 39 is an outlook view showing another rotary instrument FIG. 40 is an explanatory view for explaining the method of removing the ligament graft-fixing unit from a bone tunnel to be carried out by using the rotary instrument.

A rotary instrument 180 is a wrench. The rotary instrument 180 includes a wrench-mounting part 181 provided at a rear end portion of the instrument 180; a shaft 182 provided at a front side of the wrench-mounting part 181; and a coupling part 183 provided at a front end portion of the shaft 182 and connected with the ligament graft-fixing unit 2. The coupling part 183 has a mechanism (such as a ball joint) capable of changing an angle of coupling part 183 to the shaft 182 and connecting the coupling part 183 with the shaft 182. The front end of the coupling part 183 has a configuration capable of closely contacting with the ligament graft-holding part 8 and connecting with it Concretely the front end of the coupling part 183 is a branch-shaped portion and the coupling part 183 is connected with the ligament graft-holding part 8 with the inner surface of the branch-shaped portion closely contacting with the outer surface of the ligament graft-holding part 8.

Firstly an instrument for cutting the ligament graft 80 is inserted into a joint of knee. The ligament graft 80 mounted on the ligament graft-holding part 8 is cut with the instrument and the ligament graft 80 cut is removed from inside the bone tunnel 39. Then the wrench 180 is inserted into the bone tunnels 39a from a cut portion formed inside of the anterior surface of knee joint, and the coupling part 183 is connected with the ligament graft -holding part 8 as shown in FIG. 40. The unit 2 is rotated in a direction opposite to the bone-drilling direction. Thereby the ligament graft-fixing unit 2 can be removed from the bone tunnels 39a. Since the coupling part 183 has a mechanism capable of changing an angle of coupling part 183 to the shaft 182, even if the bone tunnels 39a and the bone tunnels 39b are not formed along the same axis, the above-described operation can be conducted with the wrench 180.

The use of the ligament graft-fixing apparatus of the present invention eliminates the need for performing additional multi-stage operational steps during placing the ligament graft-fixing unit to the predetermined position in the bone tunnel unlike the conventional art Thus it is possible to fix the ligament graft to the predetermined position in the bone tunnel by performing a simple operation. Further since one end of the ligament graft 80 can be fixed to a position inside the femur 36, the bone can be tightly fixed to the ligament graft.

Another embodiment of ligament graft-fixing apparatus of the present invention will be described below with reference to the accompanied drawings.

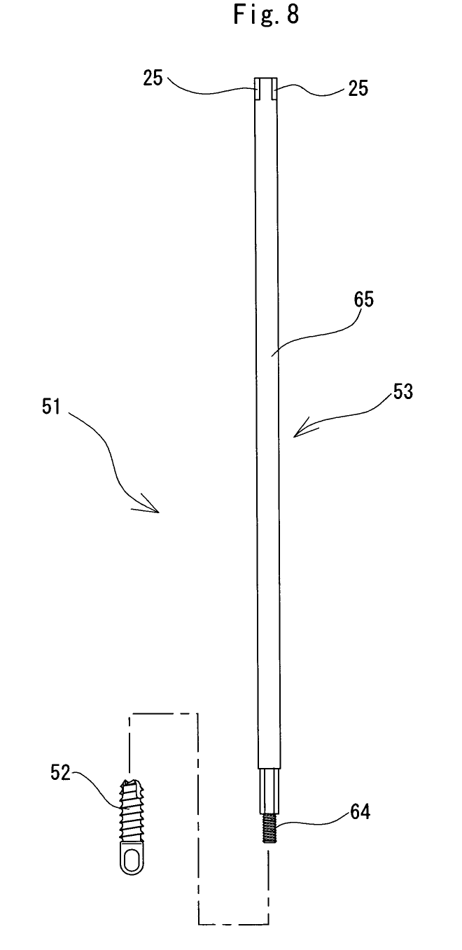

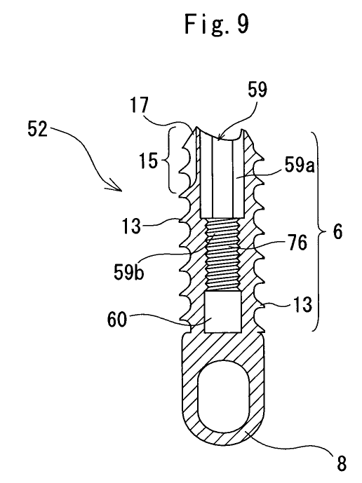

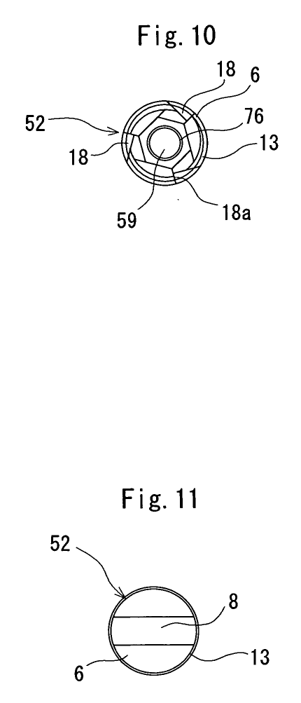

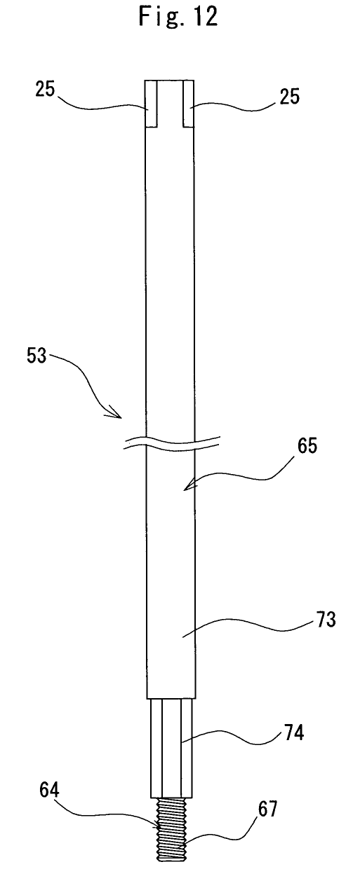

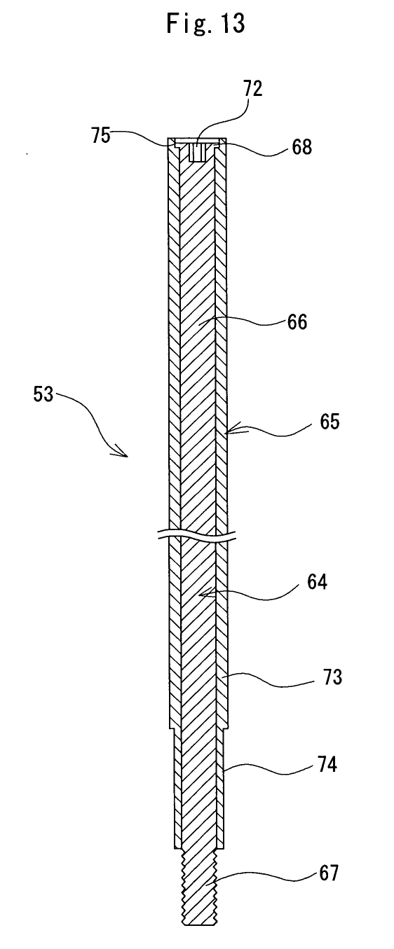

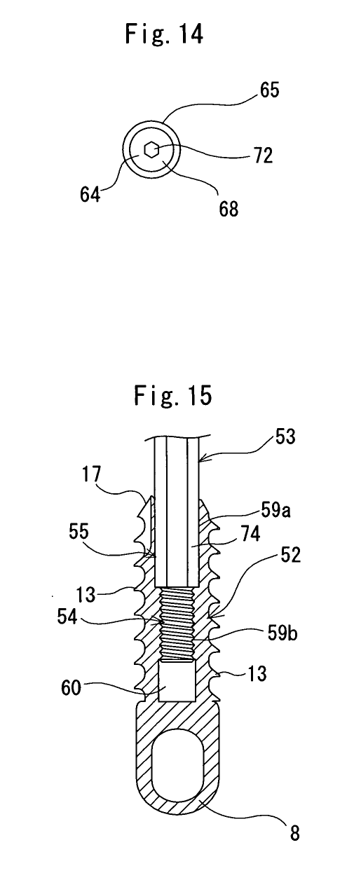

FIG. 8 is an outlook view showing an embodiment of the ligament graft-fixing apparatus of the present invention. FIG. 9 is a sectional view showing a ligament graft-fixing unit constructing the ligament graft-fixing apparatus shown in FIG. 8. FIG. 10 is a plan view showing the ligament graft-fixing unit shown in FIG. 9. FIG. 11 is a bottom view showing the ligament graft-fixing unit shown in FIG. 9. FIG. 12 is an enlarged view showing a rod-shaped shaft constructing the ligament graft-fixing apparatus shown in FIG. 8. FIG. 13 is a sectional view showing the rod-shaped shaft shown in FIG. 12. FIG. 14 is a plan view showing the rod-shaped shaft shown in FIG. 12.

A ligament graft-fixing apparatus 51 includes a ligament graft-fixing unit 52 and a rod-shaped shaft (hereinafter referred to as merely shaft) 53. The ligament graft-fixing apparatus 51 of this embodiment is different from the ligament graft-fixing apparatus 1 of the above-described embodiment in the configuration of the removal prevention mechanism 54 and the torque transmission mechanism 55. More specifically, the ligament graft-fixing apparatus 51 of this embodiment is different from the ligament graft-fixing apparatus 1 in the configuration of the mounting tunnel and that of one end portion of the shaft. The ligament graft-fixing apparatus 51 is described below mainly on the constructions different from those of the ligament graft-fixing apparatus 1.

The ligament graft-fixing apparatus 51 includes the ligament graft-fixing unit 52 and the rod-shaped shaft 53.

The ligament graft-fixing unit 52 includes a cylindrical body part 6 whose front end and rear end are open; a screw part 13 provided on a side surface of the body part 6; a cutting-edge part 15 formed at the front portion of the body part 6 to drill a bone and enlarge the diameter of the bone tunnel; a ligament graft-holding part 8 formed at the rear side of the body part 6; and a mounting tunnel 59 formed inside the body part 6. The outer configuration of the ligament graft-holding part 8, the cylindrical body part 6, the screw part 13, the cutting-edge part 15, and the ligament graft-fixing unit 52 is similar to that of the ligament graft-holding part, the cylindrical body part, the screw part, the cutting-edge part of the ligament graft-fixing unit 2, and the ligament graft-fixing unit 2 respectively. Thus description of these parts and the ligament graft-fixing unit 52 is omitted herein.

As shown in FIG. 9, the mounting tunnel 59 is formed inside the cylindrical body part 6. The mounting tunnel 59 has an accommodation tunnel 59a, almost regular hexagonal in its sectional configuration, for accommodating an insertion part of an outer tube of the shaft 53; and an accommodation tunnel 59b. The accommodation tunnel 59a is formed in the range from an opening formed at its front end to the neighborhood of its center. The accommodation tunnel 59b, continuous with the accommodation tunnel 59a, is formed downward to a position in the neighborhood of the rear end portion of the ligament graft-fixing unit 52. The accommodation tunnel 59b has a mounting tunnel-side engaging part 76, formed on the inner surface thereof, which engages a shaft-side engaging part 67 of a core 64. An inner tunnel 60 having almost the same inner diameter of the accommodation tunnel 59b (mounting tunnel 59) is formed at the rear side of the accommodation tunnel 59b. The inner diameter of the mounting tunnel-side engaging part 76 is set smaller than that of the accommodation tunnel 59a. The rear-end surface of the accommodation tunnel 59a is vertical to the axial direction of the ligament graft-fixing unit 52. In this embodiment, the inner configuration of the accommodation tunnel 59a is formed in correspondence to that of an insertion part 74 formed at one end portion of an outer tube 65. However, the accommodation tunnel 59a may take any inner configurations, provided that the side surface of the shaft 53 and that of the mounting tunnel 59 contact each other when the shaft 53 is rotated in the bone-drilling direction by the ligament graft-fixing unit 52 and that the torque is transmitted from the shaft 53 to the ligament graft-fixing unit 52.

As shown in FIGS. 12 and 13, the shaft 53 includes the core 64 and the outer tube 65 accommodating the core 64.

As shown in FIG. 13, the core 64 is solid and has a body part 66; the shaft-side engaging part 67, formed at one side of the body part 66, which engages the mounting tunnel-side engaging part 76; and a disk-shaped diameter-enlarged part 68, disposed at the other end portion of the body part 66, for preventing the core 64 from being removed forward from the outer tube 65. The body part 66 of the core 64 has almost the same diameter from its one end to its other end. The core 64 is independently sidable and rotatable inside the outer tube 65. As shown in FIGS. 13 and 14, an opening 72 for allowing the rotation of the core 64 by using a hexagonal wrench or the like is formed at the other end portion of the diameter-enlarged part 68. The core 64 can be rotated independently of the outer tube 65 by using the hexagonal wrench or the like.

As shown in FIGS. 12 and 13, the cylindrical outer tube 65 has almost the same inner diameter except the other end portion thereof. The outer tube 65 includes a body part 73; the insertion part 74 provided at one side of the body part 73; and a drill-mounting part 25, provided at the other end portion of the outer tube 65, which is mounted on a drill or the like. As shown in FIG. 13, the outer tube 65 has a length for accommodating the core 64 except the shaft-side engaging part 67, when the diameter-enlarged part 68 advances to the front with respect to the outer tube 65. The outer tube 65 is rotatable independently of the core 64.

As shown in FIG. 13, the body part 73 of the outer tube 65 has almost the same diameter in the range from its one end to its other end. Accommodation part 75 for accommodating the diameter-enlarged part 68 of the core 64 is formed in the inner tunnel 60 disposed at the other end portion of the body part 73. The accommodation part 75 for accommodating the diameter-enlarged part 68 is formed as a columnar space having a diameter larger than the diameter of the inner tunnel 60 except the inner diameter of the other end portion thereof. As shown in FIG. 13, the diameter-enlarged part 68 of the core 64 is completely accommodated in the accommodation part 75 for accommodating the diameter-enlarged part 68, when the core 64 advances to the front with respect to the outer tube 65. The body part 73 of the outer tube 65 may have a removal prevention mechanism (not shown) for preventing the core 64 from being removed from the outer tube 65 at the other end thereof. More specifically, it is preferable that the removal prevention mechanism may be composed of an engaging part formed on the inner surface of the other end portion of the body part 73 of the outer tube 65 and an engaging part formed on the outer surface of the other end portion of the core 64.

As shown in FIG. 12, the diameter of the insertion part 74 is set smaller than the outer diameter of the body part 73 of the outer tube 65. The outer configuration of the insertion part 74 is in the shape of a regular hexagon in its sectional configuration. The insertion part 74 can be accommodated in the accommodation tunnel 59a of the mounting tunnel 59 for accommodating the insertion part 74 of the outer tube 65 without forming a gap between the insertion part 74 and the accommodation tunnel 59a. The insertion part 74 may have any configurations, provided that the insertion part 74 is capable of transmitting the torque of the shaft 53 to the ligament graft-fixing unit 52.

As shown in FIG. 15, a removal prevention mechanism 54 is constituted of one end portion of the core 64 and the mounting tunnel 59. A torque transmission mechanism 55 is constituted of the outer tube 65 and the mounting tunnel 59.

The removal prevention mechanism 54 is an engaging mechanism, formed on the one end of the core 64 and the mounting tunnel 59, in which the core 64 and the mounting tunnel 59 engage each other. The torque transmission mechanism 55 is formed on the outer surface of the outer tube 65 and the side surface of the mounting tunnel 59. When the shaft 53 is rotated in the direction in which the bone is drilled by the ligament graft-fixing unit 52, the side surface of the shaft 53 and that of the mounting tunnel 59 contact each other.

The removal prevention mechanism 54 is composed of the shaft-side engaging part 67 and the mounting tunnel-side engaging part 76. The shaft-side engaging part 67 and the mounting tunnel-side engaging part 76 are threaded in the direction in which they engage each other when the shaft 53 is rotated for the ligament graft-fixing unit 52 in the bone-drilling direction. The shaft-side engaging part and the mounting tunnel-side engaging part may be threaded in the direction opposite to the direction in which they engage each other when the shaft 53 is rotated for the ligament graft-fixing unit 52 in the bone-drilling direction.

The torque transmission mechanism 55 is composed of the outer side surface of the insertion part 74 of the outer tube 65 and the inner side surface of the accommodation tunnel 59a for accommodating the insertion part of the outer tube 65.

In the above-described construction, after the insertion part 74 of the outer tube 65 is accommodated in the accommodation tunnel 59a, the opening 72 for allowing the rotation of the core 64 is rotated in the bone-drilling direction by using the hexagonal wrench or the like. Thereby as shown in FIG. 15, the shaft-side engaging part 67 of the core 64 and the mounting tunnel-side engaging part 76 engage each other. Thereby it is possible to prevent the shaft 53 from being removed from the mounting tunnel 59 while an operation of rotating the ligament graft-fixing unit 52 is being performed. After the shaft-side engaging part 67 and the mounting tunnel-side engaging part 76 are engaged each other, the rear end portion of the outer tube 65 is fixed to a grip of the drill to rotate the outer tube 65 in the bone-drilling direction. As a result, the rotational force of the outer tube 65 is transmitted to the ligament graft-fixing unit 52. Thereby the ligament graft-fixing unit 52 rotates in the bone-drilling direction. After an operation of placing the ligament graft-fixing unit 52 at the predetermined position, the core 64 is rotated in the direction in which the shaft-side engaging part 67 and the mounting tunnel-side engaging part 76 are disengaged from each other by using the hexagonal wrench or the like. Thereby the shaft-side engaging part 67 and the mounting tunnel-side engaging part 76 are disengaged from each other. Thus the shaft 53 can be easily removed from the ligament graft-fixing unit 52.

As described above, in this embodiment, the shaft-side engaging part 67 of the core 64 and the mounting tunnel-side engaging part 76 constitute the removal prevention mechanism 54. The insertion part 74 of the outer tube 65 and the accommodation tunnel 59 for accommodating the insertion part of the outer tube 65 of the ligament graft-fixing unit 52 constitute the torque transmission mechanism 55.

The method of placing the ligament graft-fixing apparatus 51 of this embodiment at a predetermined position (the method of fixing the ligament graft 80 in a bone) is similar to the method of placing the ligament graft-fixing apparatus 1 at the predetermined position except the method of operating the removal prevention mechanism 54 for preventing the shaft 53 and the ligament graft-fixing unit 52 from disengaging from each other.

It is preferable that in the method shaft 53 comprises a core 64 accommodated within and rotatable relative to an outer tube 65, the core 64 comprising a threaded end portion 67, the outer tube 65 comprising an insertion part 74 having a noncircular cross-sectional shape and the ligament graft-fixing unit 52 comprising a mounting tunnel 59 that included a first accommodation tunnel 59b that is threaded and a second accommodation tunnel 59a having a cross-sectional shape corresponding to the non-circular cross-sectional shape of the insertion portion 74 of the shaft 53, the shaft 53 being engaged with the ligament graft-fixing unit 52 by threadably engaging the threaded end portion 67 of the core 64 with the first accommodation tunnel 59b and positioning the insertion part 74 in the second accommodation tunnel 59a.

Another embodiment of ligament graft-fixing apparatus of the present invention will be described below with reference to the accompanied drawings.

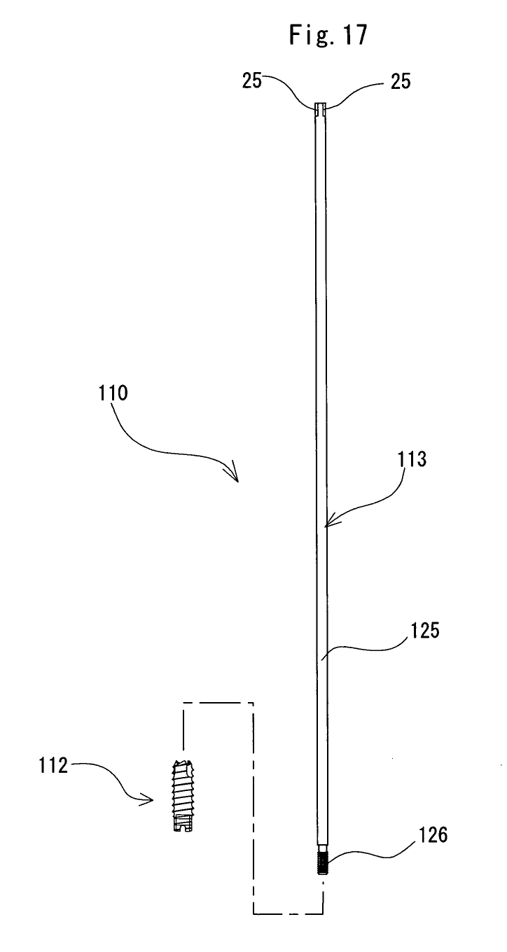

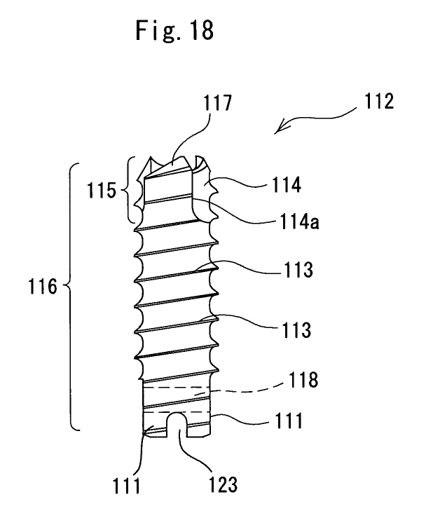

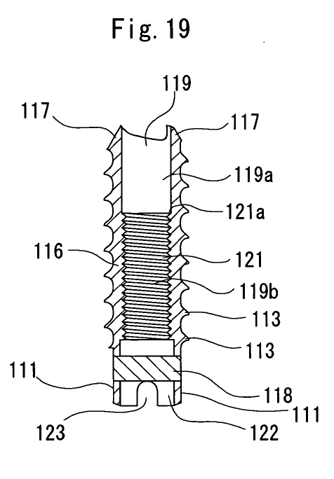

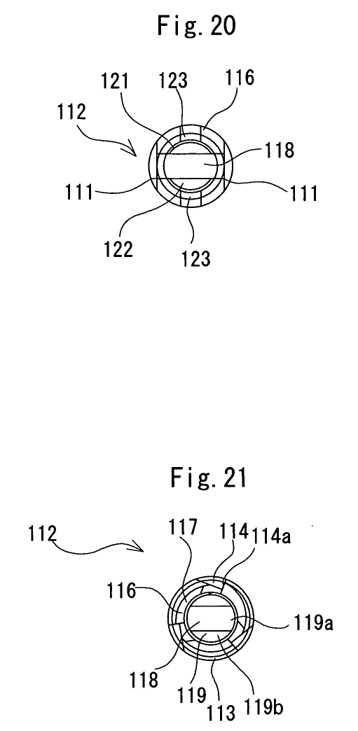

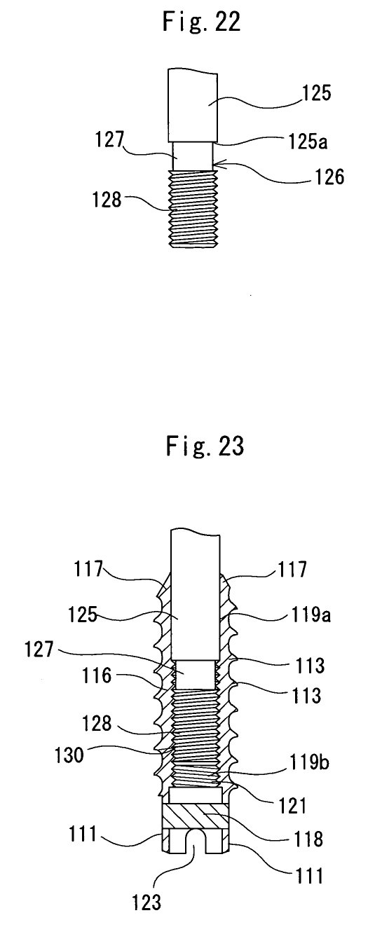

FIG. 17 is an outlook view showing still another embodiment of the ligament graft-fixing apparatus of the present invention. FIG. 18 is a front view showing a ligament graft-fixing unit constructing the ligament graft-fixing apparatus shown in FIG. 17. FIG. 19 is a sectional view showing the ligament graft-fixing unit shown in FIG. 18. FIG. 20 is a bottom view showing the ligament graft-fixing unit shown in FIG. 18. FIG. 21 is a plan view showing the ligament graft-fixing unit shown in FIG. 18. FIG. 22 is an enlarged view showing a front portion of a rod-shaped shaft constructing the ligament graft-fixing apparatus shown in FIG. 17.

A ligament graft-fixing unit 112 of this embodiment is different from the ligament graft-fixing unit 2 of the above-described embodiment in that the ligament graft-fixing unit 112 has a ligament graft-holding part 118 inside its rear end portion thereof and that a groove part 123, connected to a rotary instrument, which is used to rotate the ligament graft-fixing unit 112 is provided at the rear end portion of the ligament graft-fixing unit 112.

As shown in FIG. 17, a ligament graft-fixing apparatus 110 includes the ligament graft-fixing unit 112 and a rod-shaped shaft (hereinafter referred to as merely shaft) 113.

The ligament graft-fixing unit 112 includes a cylindrical body part 116; a screw part 113 provided on an outer surface of the body part 116; a cutting-edge part 115 formed at a front portion of the body part 116 to drill a bone and enlarge the diameter of the bone tunnel; a ligament graft-holding part 118 formed inside a rear end portion of the body part 116; a mounting tunnel 119 formed inside the body part 116; and a coupling part 123, to be connected with a drill or the like, which is formed at a rear end portion of the body part 116.

The cylindrical body part 116 is open at its front end and rear end. The screw part 113 is formed from the front end of the body part 116 to the neighborhood of its rear end. A flat surface 111 is formed on both side surfaces of the rear end portion of the body part 116.

As shown in FIG. 18, the screw part 113 is formed as a screw thread disposed on the side surface of the body part 116 from the neighborhood of the front end thereof to the neighborhood of the rear end thereof. The height of the screw thread 113 is formed almost uniform except the neighborhood of the front end of the body part 116. The height of the screw thread 113 in the neighborhood of the front end of the body part 116 is set a little smaller than that of the screw thread in other portions of the body part 116. The screw thread 113 is spaced at almost regular intervals. More specifically, the screw thread 113 is spaced at intervals of 1.40 mm to 2.50 mm and preferably 1.5 mm to 2.2 mm. The height of the screw thread 113 is set to 0.4 mm to 1.2 mm and preferably to 0.5 mm to 1.0 mm. The outer diameter of the body part 116 is set to 4.5 mm to 10.0 mm and preferably to 5.0 mm to 7.0 mm. It is preferable to set the diameter of the valley to 3.5 mm to 9.0 mm. A few kinds of ligament graft-fixing units of the above-described size are preferably prepared so that units corresponding to thickness of a harvested ligament graft can be selected. A screw part is formed at the body part 116 except the rear end of the body part 116.

As shown in FIGS. 18 and 21, the cutting-edge part 115 includes a pointed-end portion 117 formed at the front end portion of the body part 116, with the pointed-end portion 117 tapering off toward the front end of the body part 116; and cut-out portions 114 cut out from the front end of the body part 116 toward its rear end. It is preferable that the cut-out portions 114 are formed almost equiangularly about the axis of the body part 116. In this embodiment, the cut-out portions 114 are formed equiangularly at 120° about the axis of the body part 116.

In this construction, when the ligament graft-fixing unit 112 is rotated inside the bone tunnel, the inner wall of the bone tunnel is cut by a side edge 114a of the cut-out portions 114. At this time, the ligament graft-fixing unit 112 advances inside the bone tunnel owing to the operation of its screw part 113 with the ligament graft-fixing unit 112 rotating and is then firmly held inside the bone tunnel.

The mounting tunnel 119 has an accommodation tunnel 119a which is formed in almost the same diameter throughout the range from the front end of the body part 116 toward rear end of the body part 116; and an accommodation tunnel 119b, formed continuously with the rear end of the accommodation tunnel 119a, which has a mounting tunnel-side engaging part 121, engaging a shaft-side engaging part 128, formed on the inner surface of the accommodation tunnel 119b. At the rear side of the mounting tunnel 119 of the body part 116, a rear-end inner tunnel 122 is formed up to the rear end of the body part 116. The inner diameter of the accommodation tunnel 119a is set larger than that of the mounting tunnel-side engaging part 121 and almost equally to the outer diameter of the body part 125. In this construction, the engagement between the shaft 113 and the mounting tunnel 119 proceeds until a front surface of the body part 125 and a rear surface of the mounting tunnel-side engaging part 121 contact each other. The inner diameter of the rear end portion inner tunnel 122 is set a little larger than that of the accommodation tunnel 119b. The length of the mounting tunnel-side engaging part 121 is set larger than that of the shaft-side engaging part 128.

The ligament graft-fixing unit 112 has a rotary instrument coupling part 123, provided at the rear end portion of the ligament graft-holding part 118, which is connectable to a drill or the like to rotate the ligament graft-fixing unit 112. The rotary instrument coupling part 123 is a groove part (notch) 123 formed at the rear end portion of the ligament graft-fixing unit 112. One groove part 123 is formed in the range forward from the rear end of each of opposed flat surface-forming portions 111 disposed at the rear end portion of the body part 116. The groove part (notch) 123 is U-shaped. The groove part 123 may have any configurations, provided that the groove part 123 can be connected with the tip of a wrench. Three or more rotary instrument coupling parts 123 may be formed equiangularly (every 90°) about the axis of the ligament graft-fixing unit 112. In this embodiment, the cut-out portions 114 are formed equiangularly at 120° about the axis of the body part 116. For example, four rotary instrument coupling parts 123 may be formed equiangularly about the axis of the ligament graft-fixing unit 112. In an operation for reconstructing anterior or posterior crucial ligament graft of knee, the rotary instrument coupling part 123 is connected with the wrench or the like (rotary instrument) at the rear end of the ligament graft-fixing unit 112 to rotate the ligament graft-fixing unit 112. Thereby the ligament graft-fixing unit 112 can be easily taken out of the bone tunnel.

As shown in FIG. 19, the ligament graft-holding part 118 is a rod-shaped part 118 crossing the rear end portion inner tunnel 122 of the body part 116. It is preferable that the rod-shaped part 118 is placed at a position where the rod-shaped part 118 does not radially overlap the rotary instrument coupling part 123. As shown in FIG. 20, the rod-shaped part 118 is disposed almost perpendicularly to the opposed rotary instrument coupling parts 123. As shown in FIG. 19, the rod-shaped part 118 is located at a position, forward from the rotary instrument coupling part 123, at which the rod-shaped part 118 does not axially overlap the groove part 123. The rod-shaped part 118 may be located at a position where the rod-shaped part 118 does not radially overlap the rotary instrument coupling part 123 and may axially overlap the rotary instrument coupling part 123. The ligament graft can be easily mounted on the ligament graft-holding part by disposing the rod-shaped part at a position where the rod-shaped part overlaps the rotary instrument coupling part 123 axially, namely, by disposing the rod-shaped part at a position nearer to the rear end of the ligament graft-fixing unit 112. The ligament graft-holding part 118 is provided by forming an opening on both side surfaces 111 of the rear end portion of the body part 116 and inserting a pin (rod-shaped member) into the opening by press fit. The ligament graft 80 is mounted on the rod-shaped part 118 directly or indirectly through a connection member (ligature). Since the ligament graft-holding part is provided inside the rear portion of the ligament graft-fixing unit, the ligament graft-fixing unit can be advanced easily into the bone tunnel.

The ligament graft-fixing unit 112 constructing the ligament graft-fixing apparatus 110 has the cutting-edge part 115 formed at the front portion of the cylindrical body part 116; and the screw part 113 formed on the side surface of the body part 116 from the neighborhood of the front end thereof toward the rear end thereof. But it is possible that the ligament graft-fixing unit 112 does not have the cutting-edge part and has the screw part formed on the side surface of the body part 116 from the front end thereof toward the rear end thereof and that the front end portion of the ligament graft-fixing unit 112 has a diameter set a little smaller than that of the other portion thereof.

The shaft 113 is solid. As shown in FIGS. 17, 22, and 23, the shaft 113 has a rod-shaped body part 125; a front end part 126 formed at one end portion of the body part 125; and a drill-mounting part 25, provided at the other end portion of the body part 125, on which a rotational force-imparting instrument such as a drill can be mounted.

The body part 125 has an almost uniform outer diameter from its one end to its other end. The drill-mounting part 25 is formed by forming a flat surface on the rear end portion of the body part 125 every predetermined (equal) angle about the axis of the body part 125. In this embodiment, the drill-mounting part 25 is formed every 120°. As shown in FIG. 30, the shaft 113 is mounted on a grip 30 of a drill in the drill-mounting part 25.

The front end part 126 of the shaft 113 has a columnar part 127 disposed at one side of the body part 125 and having a smaller diameter than that of the body part 125; and a shaft-side engaging part 128, disposed at one side of the columnar part 127, which engages the mounting tunnel-side engaging part 121. The outer diameter of the columnar part 127 is set smaller than that of the shaft-side engaging part 128 and that of the body part 125. The outer diameter of the body part 125 is set almost equally to that of the accommodation tunnel 119a and larger than the inner diameter of the mounting tunnel-side engaging part 121. The front end of the body part 125 is formed as a flat surface. In this construction, with the shaft 113 accommodated in the mounting tunnel 119, the engagement between the shaft 113 and the mounting tunnel 119 proceeds until the front surface 125a of the body part 125 and the rear surface 121a of the mounting tunnel-side engaging part 121 contact each other. Owing to the contact therebetween, the shaft 113 is fixed to a predetermined portion inside the mounting tunnel 119. As shown in FIG. 23, the shaft 113 is accommodated in the mounting tunnel 119 up to one end portion of the body part 125 thereof.

The mounting tunnel 119 and the rod-shaped shaft 113 comprise a torque transmission mechanism which transmits a rotational force imparted to the rod-shaped shaft 113 to the ligament graft-fixing unit 112 when the rod-shaped shaft 113 is rotated in a direction in which the ligament graft-fixing unit 112 advances in the bone tunnel while the ligament graft-fixing unit 112 is mounted on the rod-shaped shaft 113; and a removal prevention mechanism which holds the ligament graft-fixing unit 112 on the rod-shaped shaft 113.

The torque transmission mechanism and the removal prevention mechanism include an engaging mechanism, formed on the shaft 113 and the mounting tunnel 119, whose engaging parts engage each other in a direction in which the shaft 113 is rotated to cause the ligament graft-fixing unit 112 to drill the bone.

The engaging mechanism 130 has the shaft-side engaging part 128 formed at a front portion of the shaft 113; and the mounting tunnel-side engaging part 121 provided on the inner surface of the mounting tunnel 119. The shaft-side engaging part 128 and the mounting tunnel-side engaging part 121 are threaded in a direction in which the shaft-side engaging part 128 and the mounting tunnel-side engaging part 121 engage each other, when the shaft 113 is rotated in the mounting tunnel 119 in the bone-drilling direction.

In the above-described construction, the shaft 113 inserted into the mounting tunnel 119 advances forward in engagement with the mounting tunnel 119 until the front surface of the body part 125 and the rear surface of the mounting tunnel-side engaging part 121 contact each other. Owing to the contact therebetween, the shaft 113 is fixed to the mounting tunnel 119. Thereafter the shaft 113 is rotated in its advance direction after the contact therebetween. As a result, a rotational force imparted to the shaft 113 is transmitted to the ligament graft-fixing unit 112. Consequently the ligament graft-fixing unit 112 advances toward the front end (to cutting-edge part 115) of the bone tunnel, while the ligament graft-fixing unit 112 is rotating in the bone-drilling direction. After the ligament graft-fixing unit 112 is placed at a predetermined position of the bone tunnel, the shaft 113 is rotated in a reverse direction. As a result, the shaft-side engaging part 128 and the mounting tunnel-side engaging part 121 disengage from each other. Thereby the shaft 113 can be removed from the mounting tunnel 119. As described above, the engaging mechanism 130 serves as the torque transmission mechanism and the removal prevention mechanism.

The torque transmission mechanism and the removal prevention mechanism may be composed of a locking mechanism and a contact mechanism instead of the engaging mechanism. It is preferable that the locking mechanism has a shaft-side locking part and a mounting tunnel-side locking part and that when the shaft is rotated in the mounting tunnel in the bone-drilling direction, the mounting tunnel-side locking part and the shaft-side locking part engage each other.

The method of placing the ligament graft-fixing apparatus 110 of this embodiment in the bone tunnel(a method of fixing the ligament graft in a bone) is similar to the method of placing the ligament graft-fixing apparatus 1 of the above-described embodiment in the bone tunnel. Thus description of the method of placing the ligament graft-fixing apparatus 110 in the bone tunnel is omitted herein.

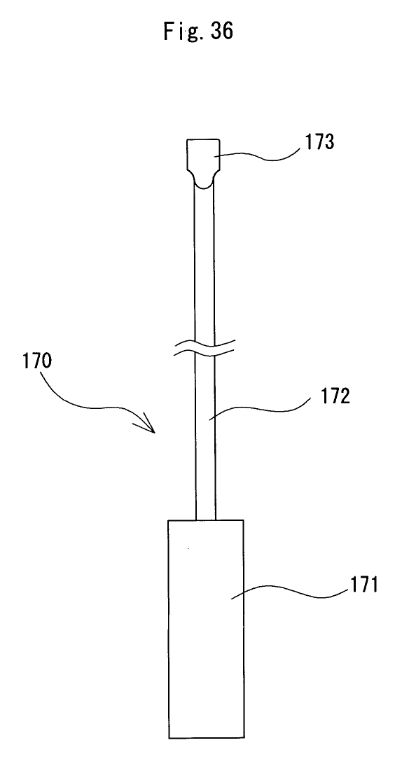

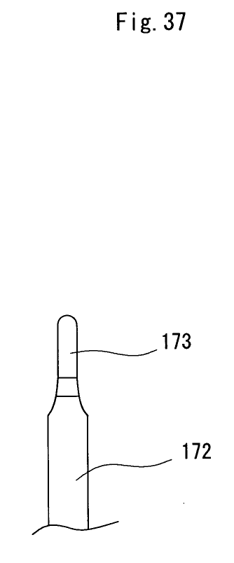

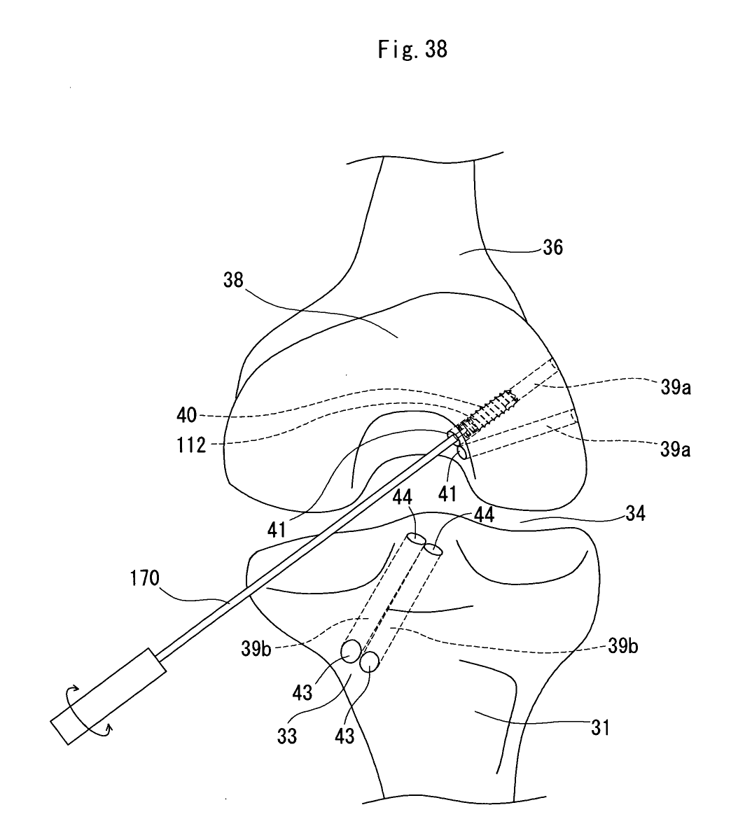

Description is made on the method of removing the ligament graft-fixing unit from a bone tunnel to be carried out by using the rotary instrument in a revision surgery for reconstructing anterior or posterior crucial ligament graft of knee. FIG. 36 is an outlook view showing the rotary instrument. FIG. 37 is an enlarged side view showing a front end portion of the rotary instrument shown in FIG. 36. FIG. 38 is an explanatory view for explaining the method of removing the ligament graft-fixing unit from a bone tunnel to be carried out by using the rotary instrument.

As shown in FIG. 36 and FIG. 37, a rotary instrument (wrench) 170 includes a wrench-mounting part 171; a shaft 172 provided at a front side of the wrench-mounting part 171; and a coupling part 173 provided at a front end portion of the shaft 172 and connected with the groove part 123. The coupling part 173 is flat and has a diameter larger than that of the shaft 172. The coupling part 173 has a configuration corresponding to that of the groove part 123 so that the coupling part 173 can be connected in close contact with the groove part 123. Although not shown, the coupling part 173 may be bifurcated, and bifurcated portions may be connected with the groove parts 123 respectively.

Initially a cutting instrument (not shown) is inserted into the opening 43 at the anterior surface-side of the tibia 31 to cut the ligament graft 80 mounted on the ligament graft-holding part 118. Thereafter the ligament graft 80 is taken out of the bone tunnel 39. After the wrench 170 is inserted into the opening 43 at the anterior surface-side of the tibia 31, the coupling part 173 and the rotary instrument coupling part 123 are connected with each other as shown in FIG. 38. Then the ligament graft-fixing unit 112 is rotated in a direction opposite to the bone-drilling direction. Thereby the ligament graft-fixing unit 112 can be removed from the bone tunnel 39.

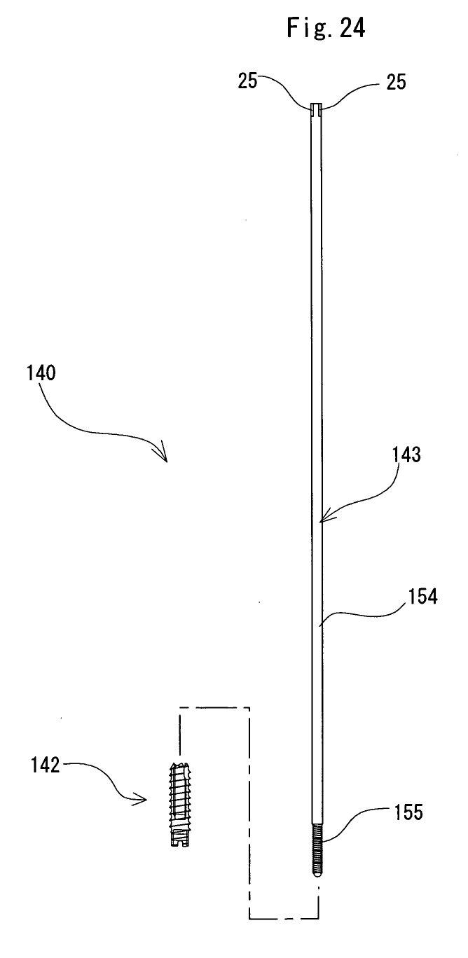

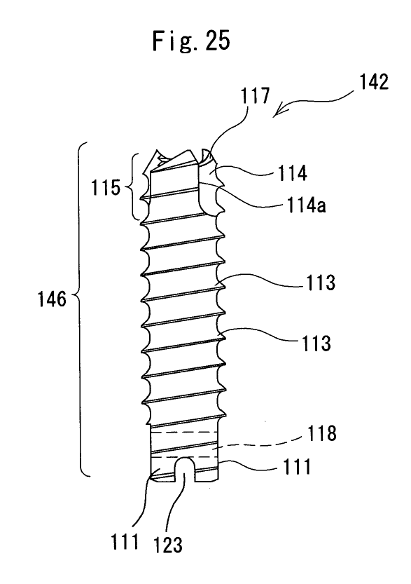

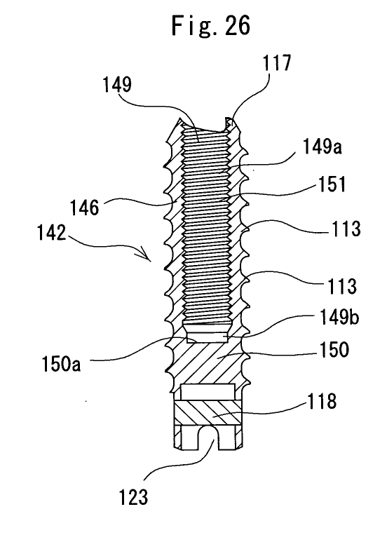

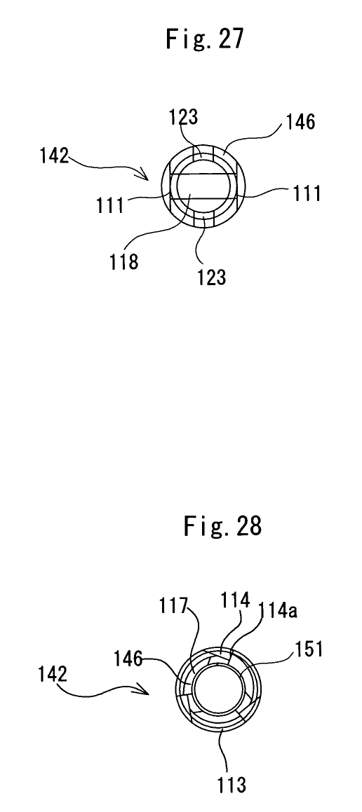

Another embodiment of the ligament graft-fixing apparatus of the present invention will be described below with reference to the accompanied drawings. FIG. 24 is an outlook view showing another embodiment of the ligament graft-fixing apparatus of the present invention. FIG. 25 is a front view showing a ligament graft-fixing unit constructing the ligament graft-fixing apparatus shown in FIG. 24. FIG. 26 is a sectional view showing the ligament graft-fixing unit shown in FIG. 25. FIG. 27 is a bottom view showing the ligament graft-fixing unit shown in FIG. 25. FIG. 28 is a plan view showing the ligament graft-fixing unit shown in FIG. 25.

A ligament graft-fixing apparatus 140 of this embodiment is different from the above-described ligament graft-fixing apparatus 110 in that an inner tunnel of a cylindrical body part 146 of a ligament graft-fixing unit 142 is closed in the neighborhood of a rear end portion of the body part 146 and that a front surface of a rod-shaped shaft (hereinafter referred to as merely shaft) 143 and a closing surface of a mounting tunnel 149 contact partly each other.

As shown in FIG. 24, the ligament graft-fixing apparatus 140 includes the ligament graft-fixing unit 142 and the shaft 143.

As shown in FIGS. 25 and 26, the ligament graft-fixing unit 142 has a cylindrical body part 146 whose front end and rear end are open and has a closing portion 150 at a rear portion of the body part 146; a screw part 113 formed on the outer side surface of the body part 146; a cutting-edge part 115 formed at the front portion of the body part 146 to drill a bone and enlarge the diameter of the bone tunnel; a ligament graft-holding part 118 formed in a rear end portion inner tunnel 152 of the body part 146; a rotary instrument coupling part 123 formed at the rear end portion of the body part 146; and the mounting tunnel 149 formed inside the body part 146. A flat surface 111 is formed on both side surfaces of the rear end portion of the body part 146. The screw part 113, the cutting-edge part 115, the ligament graft-holding part 118, and rotary instrument coupling part 123 are similar to the above-described ones respectively in the construction thereof. Thus description of these parts is omitted herein.

As shown in FIG. 26, the mounting tunnel 149 is composed of the inner tunnel 152 formed inside the body part 146 from the front end of the mounting tunnel 149 toward its rear end; and the closing portion 150. The mounting tunnel 149 has the accommodation tunnel 149a, formed in the range from the front end of the body part 146 toward its rear end, which has a mounting tunnel-side engaging part 151 formed on the inner surface thereof; and an accommodation space 149b, formed rearward from the accommodation tunnel 149a, which is capable of accommodating a contact part 157 provided at a front end portion 155 of the shaft 113 composed of the mounting tunnel 149 and a closing surface 150a. The accommodation space 149b includes a tapering part tapering off from the rear end of the accommodation tunnel 149a; and a columnar space 157 formed at the rear side of the tapering part. A front surface 150a of the closing part 150 is flat and partly contacts the contact part 157.

The ligament graft-fixing unit 142 constructing the ligament graft-fixing apparatus 140 has the cutting-edge part 115 formed at the front portion of the body part 146; and the screw part 113 formed on the side surface of the body part 146 from the neighborhood of the front end thereof toward the rear end thereof. But it is possible that the ligament graft-fixing unit does not have the cutting-edge part and has the screw part formed on the side surface of the body part in the range from the front end thereof toward the rear end thereof and that the front end portion of the ligament graft-fixing unit has a diameter set a little smaller than that of the other portion thereof.

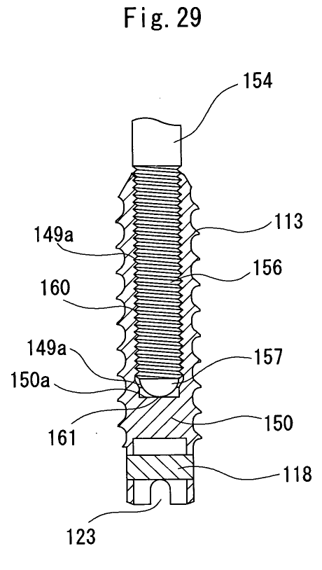

The shaft 143 is solid. As shown in FIGS. 24 and 29, the shaft 143 has a rod-shaped body part 154; a front portion 155 formed at one side of the body part 154; and a drill-mounting part 25, provided at the other end portion of the body part 154, on which a rotational force-imparting instrument such as a drill can be mounted.

The body part 154 has an almost uniform outer diameter from its one end to its other end. The drill-mounting part 25 is formed by forming a flat surface on the rear end portion of the body part 154 every predetermined (equal) angle about the axis of the body part 154. In this embodiment, the drill-mounting part 25 is formed every 120°. As shown in FIG. 30, the shaft 143 is mounted on a grip 30 of a drill in the drill-mounting part 25.

The front end portion 155 of the shaft 143 has a shaft-side engaging part 156, disposed at one side of the body part 154, which engages the mounting tunnel-side engaging part 151; and the approximately semicylindrical contact part 157 disposed forward from the shaft-side engaging part 156. The outer diameter of the shaft-side engaging part 156 is set a little smaller than that of the body part 154. As shown in FIG. 29, the shaft 143 is accommodated in the mounting tunnel 149 up to a position thereof in the neighborhood of the rear end portion of the shaft-side engaging part 156 thereof.

The mounting tunnel 149 and said rod-shaped shaft 143 comprise a torque transmission mechanism which transmits a rotational force imparted to the rod-shaped shaft 143 to the ligament graft-fixing unit 142 when the rod-shaped shaft 143 is rotated in a direction in which the ligament graft-fixing unit 142 advances in the bone tunnel while the ligament graft-fixing unit 142 is mounted on the rod-shaped shaft 143; and a removal prevention mechanism which holds the ligament graft-fixing unit 142 on the rod-shaped shaft 143.

The torque transmission mechanism includes an engaging mechanism, formed on the shaft 143 and the mounting tunnel 149, whose engaging parts engage each other in a direction in which the shaft 143 is rotated to cause the ligament graft-fixing unit 142 to drill the bone. The torque transmission mechanism also includes a contact mechanism 161 whose parts contact each other in a way preventing the shaft 143 and the mounting tunnel 149 from being tightly fixed to each other. The removal prevention mechanism comprises the engaging mechanism.

The engaging mechanism 160 has the shaft-side engaging part 156 formed at the front end portion of the shaft 143; and the mounting tunnel-side engaging part 151 provided on the inner surface of the mounting tunnel 149. The shaft-side engaging part 156 and the mounting tunnel-side engaging part 151 are threaded in a direction in which the shaft-side engaging part 156 and the mounting tunnel-side engaging part 151 engage each other, when the shaft 143 is rotated in the mounting tunnel 149 in the bone-drilling direction.

The contact mechanism 161 has a contact surface 150a formed inside the mounting tunnel 149 and the contact part 157 provided at the front end portion of the shaft 143 which contacts only a portion of the contact surface 150a. The contact surface 150a and the contact part 157 contact each other in a way preventing the rod-shaped shaft 143 and the mounting tunnel 149 from being tightly fixed to each other.

In this embodiment, the contact part 157 is formed as a rounded part 157 disposed nearer to one end of the shaft 143 than the shaft-side engaging part 156 of the front end portion 155 of the shaft 143. That is, the contact part 157 is disposed forward from the shaft-side engaging part 156. The contact surface 150a serves as a closing surface 150a disposed nearer to the rear end of the ligament graft-fixing unit 142 than the mounting tunnel-side engaging part 151 formed on the inner surface of the mounting tunnel 149. That is, the contact surface 150a is disposed rearward from the mounting tunnel-side engaging part 151, thus closing the rear end of the mounting tunnel 149. The rounded part 157 is formed approximately semispherically or approximately semi-elliptically. In this embodiment, the rounded part 157 is formed approximately semispherically. Owing to contact between the front surface of the semispherical contact part 157 and the closing surface 150a, the shaft 143 is loosely fixed to the mounting tunnel 149. Thus the shaft 143 can be easily removed from the ligament graft-fixing unit 142.

In the above-described construction, the shaft 143 inserted into the mounting tunnel 149 advances, with the shaft-side engaging part 156 in engagement with the mounting tunnel-side engaging part 151 until the front surface of the approximately semicylindrical contact part 157 disposed at the front end portion of the shaft 143 and the closing surface 150a contact each other. Owing to the contact therebetween, the shaft 143 is fixed to the mounting tunnel 149. Thereafter the shaft 143 is rotated in its advance direction. As a result, a rotational force imparted to the shaft 143 is transmitted to the ligament graft-fixing unit 142. Consequently the ligament graft-fixing unit 142 advances in the bone tunnel toward its front end (cutting-edge part 115), while the ligament graft-fixing unit 142 is rotating in the bone-drilling direction. After the ligament graft-fixing unit 142 is placed at a predetermined position in the bone tunnel, the shaft 143 is rotated in a reverse direction. As a result, the shaft-side engaging part 156 and the mounting tunnel-side engaging part 151 disengage from each other. Thereby the shaft 143 can be removed from the mounting tunnel 149. As described above, the engaging mechanism and the contact mechanism constitute the torque transmission mechanism, and the engaging mechanism constitutes the removal prevention mechanism.

The torque transmission mechanism and the removal prevention mechanism may be composed of a locking mechanism and a contact mechanism instead of the engaging mechanism. It is preferable that the locking mechanism has a shaft-side locking part and a mounting tunnel-side locking part and that when the shaft is rotated in the mounting tunnel in the bone-drilling direction, the mounting tunnel-side locking part and the shaft-side locking part engage each other.

The method of placing the ligament graft-fixing apparatus 140 of this embodiment at a predetermined position (the method of fixing the ligament graft in a bone) is similar to the method of placing the ligament graft-fixing apparatus 110 of the above-described embodiment at a predetermined position. The method of using the ligament graft-fixing unit 142 with a rotary instrument such as a drill is also similar to the method of using the ligament graft-fixing unit 112 with a rotary instrument. Thus description of the method of placing the ligament graft-fixing apparatus 140 at the predetermined position and the method of using the ligament graft-fixing unit 142 with a rotary instrument such as a drill is omitted herein.

The use of the ligament graft-fixing apparatus of the present invention eliminates the need for performing a multi-stage operation in placing the ligament graft-fixing unit to the predetermined position in the bone tunnel unlike the conventional art. Thus it is possible to fix the ligament graft to the predetermined position in the bone tunnel by performing a simple operation.

What is claimed is:

1. A ligament graft-fixing apparatus comprising:
a rod-shaped shaft; and
a ligament graft-fixing unit adapted to be removably mounted on said rod-shaped shaft to fix a ligament graft to a bone tunnel,
said ligament graft-fixing unit comprising:
a screw part provided on a side surface of said ligament graft-fixing unit which is adapted to penetrate into a bone tunnel of a bone by drilling said bone through rotation of the ligament graft-fixing unit to hold said ligament graft-fixing unit in said bone tunnel after said ligament graft-fixing unit penetrates into said bone tunnel;
a ligament graft-holding part provided at a rear end portion of said ligament graft-fixing unit to hold one of said ligament graft or a connection member mounted on said ligament graft; and
a mounting tunnel for mounting said ligament graft-fixing unit to said rod shaped shaft, said mounting tunnel being formed in said ligament graft-fixing unit from a front end thereof toward a rear end thereof, and
wherein said mounting tunnel and said rod-shaped shaft comprise:
a torque transmission mechanism which transmits a rotational force imparted to said rod-shaped shaft to said ligament graft-fixing unit when said rod-shaped shaft is rotated in a direction in which said ligament graft-fixing unit advances in said bone tunnel while said ligament graft-fixing unit is mounted on said rod-shaped shaft; and
a removal prevention mechanism which holds said ligament graft-fixing unit on said rod-shaped shaft.

2. A ligament graft-fixing apparatus according to claim 1, wherein said torque transmission mechanism and said removal prevention mechanism comprise an engaging mechanism whose engaging parts engage each other in a direction in which said rod-shaped shaft is rotated to cause said ligament graft-fixing unit to drill the bone.

3. A ligament graft-fixing apparatus according to claim 1, wherein said rod-shaped shaft comprises a core and an outer tube accommodating said core therein; said removal prevention mechanism comprises one end portion of said core and said mounting tunnel; and said torque transmission mechanism comprises said outer tube and said mounting tunnel.

4. A ligament graft-fixing apparatus according to claim 3, wherein said removal prevention mechanism is an engaging mechanism formed on one end portion of said core and said mounting tunnel in such a way that said core and said mounting tunnel engage each other; said torque transmission mechanism is formed on an outer surface of said outer tube and a side surface of said mounting tunnel, wherein when said rod-shaped shaft is rotated in a direction in which the bone is drilled by said ligament graft-fixing unit, said side surface of said rod-shaped shaft and said side surface of said mounting tunnel contact each other.

5. A ligament graft-fixing apparatus according to claim 1, wherein said torque transmission mechanism comprises an engaging mechanism whose engaging parts engage each other in a direction in which said rod-shaped shaft is rotated to cause said ligament graft-fixing unit to drill the bone; and a contact mechanism whose parts contact each other in a way preventing said rod-shaped shaft and said mounting tunnel from being tightly fixed to each other, and said removal prevention means comprises said engaging mechanism.

6. A ligament graft-fixing apparatus according to claim 5, wherein said contact mechanism has a contact surface formed inside said mounting tunnel and a contact part provided at a front end portion of said rod-shaped shaft which contacts only a portion of said contact surface, said contact surface and said contact part contact each other in a way preventing said rod-shaped shaft and said mounting tunnel from being tightly fixed to each other.

7. A ligament graft-fixing apparatus according to claim 6, wherein said contact part of said rod-shaped shaft is formed as a rounded part disposed nearer to one end of said rod-shaped shaft than an engaging part of said front end portion of said rod-shaped shaft; said contact surface being disposed nearer to a rear end of said ligament graft-fixing unit than an engaging part formed on an inner surface of said mounting tunnel, said contact surface closing a rear end of said mounting tunnel.

8. A ligament graft-fixing apparatus according to claim 1, wherein said ligament graft-fixing unit includes a cutting-edge part formed at a front portion of said body part to drill the bone and enlarge a diameter of the bone tunnel.

9. A ligament graft-fixing apparatus according to claim 1, wherein said ligament graft-holding part is a rod-shaped part crossing an inner tunnel formed at a rear end portion of said ligament graft-fixing unit.

10. A ligament graft-fixing apparatus according to claim 1, wherein said ligament graft-fixing unit has a rotary instrument coupling part, provided at a rear end portion of said ligament graft-fixing unit, which is connectable to a rotary instrument to rotate said ligament graft-fixing unit.

11. A ligament graft-fixing apparatus according to claim 10, wherein said rotary instrument coupling part is a groove part formed at said rear end portion of said ligament graft-fixing unit.

12. A method of fixing a ligament graft in a bone comprising:
positioning a one-piece ligament graft-fixing unit at a bone tunnel provided in a bone, the one-piece ligament graft-fixing unit comprising a screw part provided at a side surface and a ligament-graft-holding part holding one of a ligament graft and a connection member mounted on the ligament graft;
engaging a shaft with the one-piece ligament graft-fixing unit in a torque transmitting manner and rotating the shaft to rotate the one-piece ligament graft-fixing unit so that the one-piece ligament graft-fixing unit moves along the bone tunnel as the screw part of the one-piece ligament graft-fixing unit drills into the bone to hold the one-piece ligament graft-fixing unit in place within the bone tunnel; and
disengaging the shaft from the one-piece ligament graft-fixing unit.

13. A method according to claim 12, including a mounting tunnel provided in the one-piece ligament graft-fixing unit, the shaft engaging the one-piece ligament graft-fixing unit by mounting an end portion of the shaft in the mounting tunnel.

14. A method according to claim 12, wherein the one-piece ligament graft-fixing unit is positioned at one end of the bone tunnel and the shaft is inserted into the bone tunnel from an opposite end of the bone tunnel to engage the one-piece ligament graft-fixing unit.

15. A method according to claim 12, wherein the ligament-graft-holding part is positioned closer to one end of the ligament graft-fixing unit than on opposite end of the ligament graft-fixing unit, and the ligament graft-fixing unit is rotated by inserting a portion of the shaft into a mounting tunnel of the ligament graft-fixing unit from the opposite end of the ligament graft-fixing unit.

16. A method according to claim 12, wherein the shaft comprises a core accommodated within and rotatable relative to an outer tube, the core comprising a threaded end portion, the outer tube comprising an insertion part having a noncircular cross-sectional shape and the ligament graft-fixing unit comprising a mounting tunnel that included a first accommodation tunnel that is threaded and a second accommodation tunnel having a cross-sectional shape corresponding to the non-circular cross-sectional shape of the insertion portion of the shaft, the shaft being engaged with the ligament graft-fixing unit by threadably engaging the threaded end portion of the core with the first accommodation tunnel and positioning the insertion part in the second accommodation tunnel.

17. A ligament graft-fixing apparatus comprising:
an elongated shaft;
a unitary one-piece ligament graft-fixing unit positionable in a bone tunnel of a bone and adapted to be connected to the shaft in a connected state to move the ligament graft-fixing unit along the bone tunnel and disconnected from the shaft in a disconnected state;
said ligament graft-fixing unit comprising a mounting tunnel adapted to receive an end of the shaft in the connected state;
said shaft and said ligament graft-fixing unit comprising portions which engage one another to hold the ligament graft-fixing unit on the shaft in the connected state and to transmit torque from said shaft to said ligament graft-fixing unit in the connected state upon rotation of said shaft;
said ligament graft-fixing unit comprising a ligament graft holding part to hole one of a ligament graft and a connection member mounted on the ligament graft;
and
said ligament graft-fixing unit comprising a screw part on its outer periphery that drills into the bone of the bone tunnel during rotation of the shaft in the connected state to move the ligament graft-fixing unit along the bone tunnel and hold the ligament graft-fixing unit in the bone tunnel.
